SEQUENCE LISTING (40 sequences)

SEQ ID NO: 1 - DNA, 969 bp, Staphylococcus aureus
atgaaatcaaaaatttatatcttgctattatttctcatttttttatcagcatgcgctaatacgcgtcactctgaatccgataaaaatgtattaacagtttattctccgtatcaatcaaacttgattcgtccaattttaaatgaatttgaaaaacaagagcatgtcaaaattgaaattaaacacggatctactcaagtactgctttcaaacttgcataacgaagatttttcggagcgtggtgatgtctttatgggtggtgtgttgtcagaaacaattgatcatccagaagattttgttccctatcaagatacatctgtaacacagcaattagaggattatcgctcgaacaataaatatgttactagttttctattaatgccaacagttatagtagtgaattcagatttacaaggagatattaagattcgaggttatcaagatttattacaacctatacttaaaggtaaaattgcgtactcaaatccaaatacaacaacgacaggctatcaacatatgcgtgctatttatagcatgcatcatcgagtaagtgatgtgcatcaattccaaaaccatgcgatgcaactgtcaaagacgtctaaagtcattgaagatgttgcaaaaggtaaatattacgcaggtctaagctacgaacaagatgcacgcacatggaaaaacaaaggttatcctgtatcaatcgtttatccaattgaaggaacaatgttaaatgttgatggtattgctttagttaaaaatgcacaccctcatcctaagcgtaaaaagttagtgcaatatttaacaagccgctcagtacaacaacgattagttgcagagttcgatgccaagtcaattcgaaaggatgttagtgaacaaagcgatcagtctatcgaaaatcttaagaacatacctttaataccgaaatctaaattaccggatattccacatcataaatttttggagatgattcaatga

SEQ ID NO: 2 - DNA, 909 bp, Staphylococcus aureus
atgaattcagagtataaaaaaggcatatttttagcactcagtgcatacattctgtggggaatactacctatatattggcagttcgttgatgcaataggcgcatttgaaattttagcctttcgtattatattttcagcaatattcatgattttcatactcgcggttggacaaaaacaacgcaatgcatttcaacgagatatgaatcaattgttaggcaagcccattcagctattagcgattgtcgtagcaggctatgtcattacattaaattggggtacatttatttgggctgtaacgaacggtcacgtcctacaaacaagtttaggttattatataaatccacttgttagcattttgctcgcacttatctttttaaaagaaagattcaataaatttgaatggctagccattttattcgcattcatcggtgtattatatatgacgctcaagattggagaattcccaatcgtctctattatattagcgttatcctttggtacatacggattattgaaaaaagtagtacatattgatgccatcagcagtattacgattgaatgtattgttaccgcacctgctggactaatatacgttatttatttatggcagcaacatcagatgtcatttggattgaacatgtcatcattttggttgttattttctggtgctattacggcaataccactaatcctattctcagccggggcaaaacgtattccactttcgctaataggatttattcaatacgttggaccaacaataatgtttgtactcggcatatttgttttcaaagagccttttagtatagatcaattaattacgtttatatttatttggacaggtattgtgttatatagtctttctcaatacattaaattgaagaaacatccggtcgcaaaaaccctataa

SEQ ID NO: 3 - DNA, 516 bp, Staphylococcus aureus
atgactaactttacttttgatggtgcacacagtagtttagaattccaaattaaacatttaatggtttctaaagtgaaaggttcatttgatcaatttgatgtagctgttgaaggagatattaatgacttcagtactttgaaagctactgcaacaattattccaagctcaattaacactaaaaacgaagcacgtgataaccacttaaaatctggtgatttctttggtactgacgaatttgataaaattacatttgtgacaaaatcagtatctgaaagcaaagttgttggtgatttaacaattaaaggcatcactaacgaagaaacattcgatgttgaattcaacggagtaagtaagaatcctatggatggttctcaagtaacaggtattattgttactggtacaatcaatagagaaaaatatggcattaactttaaccaagcacttgaaactggtggcgtaatgctaggcaaagatgttaaattcgaagcatcagctgaattctcaatctcagaataa

SEQ ID NO: 4 - DNA, 378 bp, Staphylococcus aureus
atggataaacaagttagaaatacaacagaaattgtacgtttggcgaagcagaaatcaaaaaagacaagggaaaaagtagacaaagcgatttctaaattttcgattgaaggtaaagttattaattttaattcaatagcaaaggaagctaatgtttctaaatcatggctttataaggaacacgatattaggcaaagaatcgaatcccttcgtgagcgtcaaataacagcaaatgtagtctcaaaacccaagaaaagttctcgttcggaggaaatccttattaaaaccttaaaaagaagagtaatggaattagaaaaagaaaataaaaaattacagaaccaaattcaaaaattatatggagatctgtataataaagaataa

SEQ ID NO: 5 - DNA, 837 bp, Staphylococcus aureus
atggatgaacaacaatggactgggcaacttgatttaacagtgtttttcgatggcaatcgatcagtatcaagagatattttctttgaaaaagcacttaaagtgatacgtccagtttatctaaatcaatctaccattcctacattttatatagtaaatgtaggtggtggctatttagatggagatcgttaccgtatgaatgtgaatgtcgaagataacgctaaagtgacattgacatctcaaggtgcaacaaaaatatacaagacaccttccaatcatgttgagcagtatcaaacttttaatttgaaagataacgcatatttagaatatgtcgctgatccaatcatcgcatatgaaaatgctaaattttatcaacacaatacgttcaatctcaataattctagttcattattttatactgatattttaactcctggttattcaaaaactggcgaagccttcaagtatcaatatatgcatttaataaatgaaatttatattgaagatgagttagtcacatatgataatttattattgaatcctaataaacaatcgatcaatgaaataggttatatggaacattactctcattatggctctgcttacttcatacacgaagatgttaaccaaaaactaatcgattcagtttatgaaaccattagttcttatagcaatacattcgattgtcgtgttgctatttctcaattgcctacacatggttttgcagttagaatttttgcttatcgcacacaaattatagagaaaatacttggaaccatccaaagctatatcgcagaaaatatttatgatcgtaaacttgattttctaagaaaatattaa

SEQ ID NO: 6 - DNA, 426 bp, Staphylococcus aureus
atgaaactaaaatcatttgttactgccactttagcattgggattattatcaacggtcggagctgcattaccgagtcacgaagcatctgcagatagtaataacggctataaagaaatgactgtggatggttatcacactgttccttacacaatttcagtagatggtattactgcattacatcgaacttactttatcttcccagaaaataaaaatgttctttatcaagaaattgacagtaaagtaaaaaatgaattagcttctcaacgtggtgttacaacagaaaaaattaataatgcccaaacagcaacttatacgcttactttgaatgatggtaataaaaaagtagtgaatctaaagaaaaatgacgacgctaaaaattcaattgatccaagtacaatcaaacagatacaaattgtagttaaataa

SEQ ID NO: 7 - DNA, 516 bp, Staphylococcus aureus
atgaagattgcattaggatgcgaccatattgttacagatacaaaaatgcgtgtatctgaatttttaaaatcaaaaggacatgaagtcattgacgtaggaacatacgatttcacaagaacacattatccaatttttggtaaaaaagttggcgaacaagttgttagcggtaatgcagacttaggtgtttgtatttgtggaacaggtgttggtattaacaatgctgtaaataaagtacctggcgttcgttcagcactagtacgtgatatgacatcagcgttatacgctaaagaagaattaaatgcgaacgttattggcttcggtggacgtattataggtgagttattaatgtgcgatattatcgatgcatttattaatgctgaatataaaccaactgaagagaacaaaaaattaatcgctaaaattaaacatttagaaacaagcaatgcagatcaagctgatccacatttctttgatgaattcttagaaaaatgggacagaggcgaataccacgattaa

SEQ ID NO: 8 - DNA, 582 bp, Staphylococcus aureus
atgaatacaaaatttttaggtaaaacattagtagcaagtgctttagtattaacaacattaggaacaggtttacattcttcatacttaggattaaatacaaataaagttgttaaaacagcaaaagcagaagaaaaaatgacaaatggtcaattgtggaaaaaagttaaagattcattaattgattcaaatattattagtggtaacgaaaatgaagagattacagtaacatatgttaataaaaccggatattcttcaagtgtttctgcctatggtaataataatgacgatttttcaagtactccaagtaatttttctaaacttaaggaaattgacttgaaaaaagataatgtaccgtcagatgattttaatactactgttagtggtgaagatagttggaaaacacttacatctaaattaaaggaaaaaggtttggttacagatggacaaacagtaactattcattgtaatgataagtctgacaatacaaaatcatctgtttcaggtaaagtaggagcagatctgacaagtggcaatggaactacattcaaaaaacgctttattgataaaatcacaattgattaa

SEQ ID NO: 9 - DNA, 906 bp, Staphylococcus aureus
atgacgaatcaagacaacaatcatcaattgaatcatcgtatatatcattttgaaaagatatataaagctatcaaacatgtcattgtttttatatttatgattttcattgccatcgttgctatcgctgtgattgcgatgtctttatattttcatcatttaactaaaacgtccgactcattatcagatgatgctttaataaaaaaagttcgacaaatacctggcgatgaattattagatcataataacaaaaatttattatatgagtataaccattctcaaaactcactcattataggccctaaaacatcaagtccaaatgtcattaaagcattaacgtcatctgaagacactttattttataaacatgatggcatcttaccaaaggcgattttaagagcaatgatacaagatatttttaatactgatcaaagttcaggtggtagcacaattacacaacaacttgttaaaaatcaagttcttaccaacgaaaaaacatatagtagaaaagcaaatgaacttcgcctagcaattagattagaacacctactctcaaaagatgaaattatatatacatatttaaatatagttcccttcggtagagattataatggcgctaatatttccggaattgcatccgcttcatatagtctatttggtattccaccaaaagatttatcaattgcacaatctgcataccttatcggtttgttgcaaagcccatatggctatacaccctacgaaaaagatggaacgttaaaatcggataaagatttgaaatatagtattcaaagacaacattatgtattaaagcgtatgttaatcgaagatcaaatcactgaaaaagaatacaacgacgcattaaaatatgatattaaatcacatttgttaaatcgaaaaaagcgttaa

SEQ ID NO: 10 - DNA, 213 bp, Staphylococcus aureus
attttgagtttcactcgaatgtcagttcgaggaataaataaagttaaacgagagctaggttttgtattaatggcacttaatataaggaaaatagcagctcaacgagctgtacattataaaatacatatcaaaaaagctgatttctatcaaataattaatagaaatcagctttttacattgcctaagaacttaatgtcccagcctccttcataa

SEQ ID NO: 11 - DNA, 924 bp, Staphylococcus aureus
atggcaaatttacaaaagtatattgagtattctcgagaagttcagcaagcacgggagaacaatcaaccgattgtagcattagaatcaacaattatttcgcatggtatgccgtacccacaaaatgttgaaatggcaacaacagtagagcaaattatcaggaataatggtgccattccagcaaccatagccattatagatggcaaaattaaaattggtttagaaagcgaagatttagaaatactggcaactagtaaagacgttgctaaagtatctagaagggatttagcagaagttattgcgatgaagtgtgttggtgctactactgtagcgacgacgatgatatgtgctgcaatggctggtattcaattttttgttacaggaggtattgggggcgtccataaaggtgcagaacatacgatggacatttcagcagacttagaagaactgtctaaaacaaatgtcactgttatctgtgcaggtgccaaatcaattttagacttacctaagacgatggagtatttagaaacaaaaggcgttccagttattggatatcaaacgaatgaattgccagcattcttcactcgcgaaagcggtgttaagttaacaagttcggttgaaacgccagaacgacttgctgacattcatttaacaaaacagcagttaaatcttgaaggtggcattgttgttgctaatccaattccatatgagcatgccttatcaaaagcatatattgaggcaatcataaatgaagctgttgttgaagcggaaaatcaaggtattaaaggtaaggacgccacaccgttcttgttagggaaaattgtagaaaaaacgaatggtaaaagtttagcagcaaatataaaacttgttgaaaacaatgcggcgttgggtgctaaaattgctgtcgctgttaataaattattgtag

SEQ ID NO: 12 - DNA, 573 bp, Staphylococcus aureus
ttgacaagctttatctataaaatactttatgttgtcaaaatcaacgcttatacatatgacataatgacggaggacatcatgattctatctatattacttatctttttctgtatcagacttgtcagcttaaagatatctattaatcactcaaaacaattaaaagcagacggtgcagttgaatatggcgttaaaaattcgaagtttttagcaataacacacgttttaatttatgtattggctggtgtagaggcatttattaataaagatacatttagctttgcaaatggtattggcttagttatattaatctttgcttatatcatgttatttatggttattaaaactttaggtggtatttggacattaaaattattcattttacctaaccatcctattattaaatctggattatataaaattacaaaacacccaaattacttcttaaacatcattcctgaattaatcggtgtattattattaacacacgcaacatatacaactattttattagtaccgtatgcgtatttcttatatgtacgtattaaacaagaagagaaattaatgaatatttaa

SEQ ID NO: 13 - DNA, 1011 bp, Staphylococcus aureus
atgaatagagagatgttgtatttaaatagatcagatattgaacaagcgggaggtaatcattcacaagtttatgtggacgcattaacagaagcattaacagcccatgcgcacaatgattttgtacaaccgcttaagccgtatttaagacaggatcctgaaaatggacacatcgcagatcgaattattgcaatgccaagtcatatcggtggtgaacacgcaatttcaggtattaagtggataggtagtaagcacgacaatccatcgaaacgtaatatggagcgtgcaagtggcgtcattattttgaatgatccagaaacgaattatccaattgcagttatggaagcaagtttaattagtagtatgcgtactgcagcagtttcagtgattgcagcaaagcatttggctaaaaaaggatttaaagacttaacaatcattggatgcgggctaatcggagacaagcaattacaaagtatgttagagcaattcgatcatattgaacgcgtgtttgtttacgatcaattctctgaagcatgtgcacgctttgttgatagatggcaacaacagcgtccggaaattaattttattgcgacagaaaatgctaaagaagcagtatcaaatggtgaagtagtcattacatgtaccgtaacggatcaaccatacattgaatatgattggttacaaaagggtgcatttattagcaacatttctatcatggatgtgcataaagaagtctttattaaagctgacaaagtcgtagtagatgactggtcacaatgtaatcgagaaaagaaaactattaaccaattggtgttagaaggtaaattcagcaaagaagctcttcatgctgaactaggacaacttgtgacaggtgacataccaggacgtgaagacgatgatgagatcatattacttaatccgatgggtatggctatcgaagatatttcaagtgcttattttatttatcaacaggcacaacaacaaaatattgggacaacattgaacctatattaa

SEQ ID NO: 14 - DNA, 2373 bp, Staphylococcus aureus
gtggcaattaagaaaaaagatagaattattggagttaaagaattagaaataccgcaagaattaaagttagtgcctaattgggtattatggcgagctgaatggaacgagaagcaacaaaattatggaaaagtaccatatagtattaatggttacagagctagtacaaccaataaaaaaacatggtgtgactttgaaagtgtaagtattgaatatgaagttgatgagcaatatagcggtataggttttgtattaagtgatggtaataattttgtttgcctagatattgataatgcaattgatgaaaaaggacaaatcaattctgaattagcattaaaaatgatgcgactcacatattgtgaaaagtctccaagtggtacaggattacattgtttctttaaaggtaaactaccagataaccgtaaaaagaaaagaacggatttagacatagagttatatgattcagcaaggtttatgactgttacaggatgcacaattggtcaaaatgatatttgtgataatcaggaagtattaaatactctcattgatgaatactttaaagagaatttgccagtaaatgatgttgtgagagaggaatctaatactaatatgcaattatctgatgaagatattataaacattatgatgaaatctaaacaaaaagataaaattaaagatcttttacaaggcacatatgaatcatattttgacagttcgagcgaagcagtacaaagcttattacattatttagcgttttacacaggtaaaaacaaacagcaaatggagcgtatatttttaaactacaataatcttacggataaatgggaaagtaaacgaggtaatacgacttggggacagcttgagttagataaagctataaagaatcaaaagacagtttacactaaatccatagatgaatttaatgttatacaacaggggagtaaagatgttaaacagttattgaatcaattggggcatgaagaaagaacaaaaatggaagaaaagtggattgaaggaggaaaacgagggcgaaagcctacaacaattagccctataaaatgtgcatatattttgaatgagcatttaacatttatactttttgatgatgaagaaaatactaagttagctatgtatcaatttgatgaagggatatatacacagaacactacaattataaaacgagtaatttcctatttagagcccaaacataatagtaataaagctgatgaagttatttatcatttaaccaatatggtagatataaaagagaaaactaactcaccatacttaataccagttaaaaatggtgtatttaaccgtaaaacgaagcaactagaatcatttacacctgattgtatatttaccactaaaatagatacatcgtatgtaaggcaagatatagtacctgaaataaatggctggaatatagatcggtggatagaagaaatagcttgtaatgataatcaggttgttaaattattgtggcaagtaattaatgactcaatgaatggaaactacacacgtaaaaaagcaatatttttggttggtaatggtaacaatggtaaaggtacatttcaagaattattgtctaatgtaataggttatagcaatattgctagcttaaaagtgaatgagtttgatgaacgttttaaattgagtgtgttagagggcaagacagcagtaattggcgacgatgtaccagttggtgtgtatgtcgatgattcttcaaactttaaaagtgtagttactggcgatccagtgttggttgagtttaaaaataaacccttatatagagcgacttttaagtgtacagttattcaatcaacgaatggaatgcctaaatttaaagacaaaacaggcgggactttaagaaggttattgatagtaccgtttaatgccaattttaatggcattaaagagaattttaaaatcaaagaagactatataaaaaatccgcaagtgctagagtatgtgctttataaagcaattaatttagattttgaaacttttgacattcctgatgcatctgaaaaaatgcttgaagtatttaaagaagataacgatccagtttatgggtttaaagtaaatatgtttgatcaatggactattagaaaagtgccgaaatatattgtatacgcattttataaagaatattgtgatgaaaatggctataatgcattgagttcaaacaagttttataaacaatttgaacattatttagagaattattggaaaactgatgcacagcgaagatatgacaatgaagaacttgctaagaggatatacaactttaatgacaatagaaattacattgaacctattgaaagtggaaaaaactataaatcgtatgaaaaggtgaagctaaaagcaatatag

SEQ ID NO: 15 - DNA, 1179 bp, Staphylococcus aureus
atgaggaaagttttaaatatttttacaatagcttttaaatcaatattgaaaaataaaggtagaaatatatttacaatgataggtataatcatcggtatttcttctgttattacgataatgtctttgggaaatggctttaaaaaaactgctgctgatcaattttcagatgctggcgccggaaaacaagaagcgttgattagctttacttttaaagttgatgagaaaatcaaaaagtatccttttaatcaaagagatatagagttggtaaatcaagttgatggcgtactagatgcgaagctaaaggaaaataaagaagaaggaattgaagccacaataactaatgtccagaaaaaaagtgacatcttcattataaaaaaacaaaatttacactcttttaaagtaggtagagggtttgataaagaggataatgaattacgtaagaaaatagtggttataaatgatcaagtagcaaaaactgtattcaataataatgctattggtaaatcgctatatattgagggacagggatttgaagtcataggaattacggataaactatattctgactcatctaccgttataatgcctgaaaatacattcaactactatatgggacacttacatcaaggtctgcctaccttacagataattattgaagatggttacaataaaaaaactgtagttaaaaaagtagaatcactattaaataaaaaaggatcgggctctgttttaggtgagtatacatatacagacactgaagaaattatcaaaagtattgataaaatttttgatagtattacttattttgtagcagctgttgccggtatttcactttttattgctggtattggagttatgaatgtcatgtatatatctgtagctgaaagaacagaagaaattgcgatacgacgtgcatttggtgcaaaaagtcgagatattgaactacaatttttaatagagagcattttaatatgtgtgacaagtggttttattggacttattttaggtgttgtgtttgcaacaataattgatgtattaacaccagattatattaaaagtgtagtaagtttgagttctgtcataattgcggttagtgtgtcgatattaattggacttctttttggatggataccagctcgcgcagcatctaagaaagagctcatagatattataaaatag

SEQ ID NO: 16 - DNA, 210 bp, Staphylococcus aureus
ttgagtttcactcgaatgtcagttcgaggaataaataaagttaaacgagagctaggttttgtattaatggcaattaatataaggaaaatagcagctcaacgagctgtacattataaaatacatatcaaaaaagctgatttctatcaaataattaatagaaatcagctttttacattgcctaagaacttaatgtcccagcctccttcataa

SEQ ID NO: 17 - DNA, 294 bp, Staphylococcus aureus
atggcaatgattaagatgagtccagaggaaatcagagcaaaatcgcaatcttacgggcaaggttcagaccaaatccgtcaaattttatctgatttaacacgtgcacaaggtgaaattgcagcgaactgggaaggtcaagctttcagccgtttcgaagagcaattccaacaacttagtcctaaagtagaaaaatttgcacaattattagaagaaattaaacaacaattgaatagcactgctgatgccgttcaagaacaagaccaacaactttctaataatttcggtttgcaataa

SEQ ID NO: 18 - protein, 322 aa, Staphylococcus aureus
MKSKIYILLLFLIFLSACANTRHSESDKNVLTVYSPYQSNLIRPILNEFEKQEHVKIEIKHGSTQVLLSNLHNEDFSERGDVFMGGVLSETIDHPEDFVPYQDTSVTQQLEDYRSNNKYVTSFLLMPTVIVVNSDLQGDIKIRGYQDLLQPILKGKIAYSNPNTTTTGYQHMRAIYSMHHRVSDVHQFQNHAMQLSKTSKVIEDVAKGKYYAGLSYEQDARTWKNKGYPVSIVYPIEGTMLNVDGIALVKNAHPHPKRKKLVQYLTSRSVQQRLVAEFDAKSIRKDVSEQSDQSIENLKNIPLIPKSKLPDIPHHKFLEMIQ

SEQ ID NO: 19 - protein, 302 aa, Staphylococcus aureus
MNIEYKKGIFLALSAYILWGILPIYWQFVDAIGAFEILAFRIIFSAIFMIFILAVGQKQRNAFQRDMNQLLGKPIQLLAIVVAGYVITLNWGTFIWAVTNGHVLQTSLGYYINPLVSILLALIFLKERFNKFEWLAILFAFIGVLYMTLKIGEFPIVSIILALSFGTYGLLKKVVHIDAISSITIECIVTAPAGLIYVIYLWQQHQMSFGLNMSSFWLLFSGAITAIPLILFSAGAKRIPLSLIGFIQYVGPTIMFVLGIFVFKEPFSIDQLITFIFIWTGIVLYSLSQYIKLKKHPVAKTL

SEQ ID NO: 20 - protein, 171 aa, Staphylococcus aureus
MTNFTFDGAHSSLEFQIKHLMVSKVKGSFDQFDVAVEGDINDFSTLKATATIIPSSINTKNEARDNHLKSGDFFGTDEFDKITFETKSVTENKVVGDLTIKGITNEETFDVEFNGVSKNPMDGSQVTGVIVTGTINRENYGINFNQALETGGVMLGKDVKFEASAEFSISE

SEQ ID NO: 21 - protein, 125 aa, Staphylococcus aureus
MDKQVRNTTEIVRLAKQKSKKTREKVDKAISKFSIEGKVINFNSIAKEANVSKSWLYKEHDIRQRIESLRERQITANVVSKPKKSSRSEEILIKTLKRRVMELEKENKKLQNQIQKLYGDLYNKE

SEQ ID NO: 22 - protein, 214 aa, Staphylococcus aureus
MNVNVEDNAKVTLTSQGATKIYKTPSNHVEQYQTFNLKDNAYLEYVADPIIAYENAKFYQHNTFNLNNSSSLFYTDILTPGYSKTGEAFKYQYMHLINEIYIEDELVTYDNLLLNPNKQSINEIGYMEHYSHYGSAYFIHEDVNQKLIDSVYETISSYSNTFDCRVAISQLPTHGFAVRIFAYRTQIIEKILGTIQSYIAENIYDRKLDFLRKY

SEQ ID NO: 23 - protein, 141 aa, Staphylococcus aureus
MKLKSFVTATLALGLLSTVGAALPSHEASADSNNGYKELTMDGKHTVPYTISVDGITALHRTYFVFPENKKVLYQEIDSKVKNELASQRGVTTEKINNAQTATYTLTLNDGNKKVVNLKKNDDAKNSIDPSTIKQIQIVVK

SEQ ID NO: 24 - protein, 170 aa, Staphylococcus aureus
MKIALGCDHIVTDTKMRVSEFLKSKGHEVIDVGTYDFTRTHYPIFGKKVGEQVVSGNADLGVCICGTGVGINNAVNKVPGVRSALVRDMTSALYAKEELNANVIGFGGRIIGELLMCDIIDAFINAEYKATEENKKLIAKIKHLETSNADQADPHFFDEFLEKWDRGEYH

SEQ ID NO: 25 - protein, 193 aa, Staphylococcus aureus
MNTKFLGKTLVASALVLTTLGTGLHSSYLGLNTNKVVKTAKAEEKMTNGQLWKKVKDSLIDSNIISGNENEEITVTYVNKTGYSSSVSAYGNNNDDFSSTPSNFSKLKEIDLKKDNVPSDDFNTTVSGEDSWKTLTSKLKEKGLVTDGQTVTIHCNDKSDNTKSSVSGKVGADLTSGNGTTFKKRFIDKITID

SEQ ID NO: 26 - protein, 301 aa, Staphylococcus aureus
MTNQDNNHQLNHRIYHFEKIYKAIKHVIVFIFMIFIAIVAIAVIAMSLYFHHLTKTSDSLSDDALIKKVRQIPGDELLDHNNKNLLYEYNHSQNSLIIGPKTSSPNVIKALTSSEDTLFYKHDGILPKAILRAMIQDIFNTDQSSGGSTITQQLVKNQVLTNEKTYSRKANELRLAIRLEHLLSKDEIIYTYLNIVPFGRDYNGANISGIASASYSLFGIPPKDLSIAQSAYLIGLLQSPYGYTPYEKDGTLKSDKDLKYSIQRQHYVLKRMLIEDQITEKEYNDALKYDIKSHLLNRKKR

SEQ ID NO: 27 - protein, 70 aa, Staphylococcus aureus
MLSFTRMSVRGINKVKRELGFVLMALNIRKIAAQRAVHYKIHIKKADFYQIINRNQLFTLPKNLMSQPPS

SEQ ID NO: 28 - protein, 307 aa, Staphylococcus aureus
MANLQKYIEYSREVQQARENNQPIVALESTIISHGMPYPQNVEMATTVEQIIRNNGAIPATIAIIDGKIKIGLESEDLEILATSKDVAKVSRRDLAEVVAMKCVGATTVATTMICAAMAGIQFFVTGGIGGVHKGAEHTMDISADLEELSKTNVTVICAGAKSILDLPKTMEYLETKGVPVIGYQTNELPAFFTRESGVKLTSSVETPERLADIHLTKQQLNLEGGIVVANPIPYEHALSKAYIEAIINEAVVEAENQGIKGKDATPFLLGKIVEKTNGKSLAANIKLVENNAALGAKIAVAVNKLL

SEQ ID NO: 29 - protein, 190 aa, Staphylococcus aureus
MTSFIYKILYVVKINAYTYDIMTEDIMILSILLIFFCIRLVSLKISINHSKQLKADGAVEYGVKNSKFLAITHVLIYVLAGVEAFINKDTFSFANGIGLVILIFAYIMLFMVIKTLGGIWTLKLFILPNHPIIKSGLYKITKHPNYFLNIIPELIGVLLLTHATYTTILLVPYAYFLYVRIKQEEKLMNI

SEQ ID NO: 30 - protein, 336 aa, Staphylococcus aureus
MNREMLYLNRSDIEQAGGNHSQVYVDALTEALTAHAHNDFVQPLKPYLRQDPENGHIADRIIAMPSHIGGEHAISGIKWIGSKHDNPSKRNMERASGVIILNDPETNYPIAVMEASLISSMRTAAVSVIAAKHLAKKGFKDLTIIGCGLIGDKQLQSMLEQFDHIKRVFVYDQFSEACARFVDRWQQQRPEINFIATENAKEAVSNGEVVITCTVTDQPYIEYDWLQKGAFISNISIMDVHKEVFIKADKVVVDDWSQCNREKKTINQLVLEGKFSKEALHAELGQLVTGDIPGREDDDEIILLNPMGMAIEDISSAYFIYQQAQQQNIGTTLNLY

SEQ ID NO: 31 - protein, 790 aa, Staphylococcus aureus
MAIKKKDRIIGVKELEIPQELKLVPNWVLWRAEWNEKQQNYGKVPYSINGYRASTTNKKTWCDFESVSIEYEVDEQYSGIGFVLSDGNNFVCLDIDNAIDEKGQINSELALKMMRLTYCEKSPSGTGLHCFFKGKLPDNRKKKRTDLDIELYDSARFMTVTGCTIGQNDICDNQEVLNTLIDEYFKENLPVNDVVREESNTNMQLSDEDIINIMMKSKQKDKIKDLLQGTYESYFDSSSEAVQSLLHYLAFYTGKNKQQMERIFLNYNNLTDKWESKRGNTTWGQLELDKAIKNQKTVYTKSIDEFNVIQQGSKDVKQLLNQLGHEERTKMEEKWIEGGKRGRKPTTISPIKCAYILNEHLTFILFDDEENTKLAMYQFDEGIYTQNTTIIKRVISYLEPKHNSNKADEVIYHLTNMVDIKEKTNSPYLIPVKNGVFNRKTKQLESFTPDCIFTTKIDTSYVRQDIVPEINGWNIDRWIEEIACNDNQVVKLLWQVINDSMNGNYTRKKAIFLVGNGNNGKGTFQELLSNVIGYSNIASLKVNEFDERFKLSVLEGKTAVIGDDVPVGVYVDDSSNFKSVVTGDPVLVEFKNKPLYRATFKCTVIQSTNGMPKFKDKTGGTLRRLLIVPFNANFNGIKENFKIKEDYIKNPQVLEYVLYKAINLDFETFDIPDASEKMLEVFKEDNDPVYGFKVNMFDQWTIRKVPKYIVYAFYKEYCDENGYNALSSNKFYKQFEHYLENYWKTDAQRRYDNEELAKRIYNFNDNRNYIEPIESGKNYKSYEKVKLKAI

SEQ ID NO: 32 - protein, 392 aa, Staphylococcus aureus
MRKVLNIFTIAFKSILKNKGRNIFTMIGIIIGISSVITIMSLGNGFKKTAADQFSDAGAGKQEALISFTFKVDEKIKKYPFNQRDIELVNQVDGVLDAKLKENKEEGIEATITNVQKKSDIFIIKKQNLHSFKVGRGFDKEDNELRKKIVVINDQVAKTVFNNNAIGKSLYIEGQGFEVIGITDKLYSDSSTVIMPENTFNYYMGHLHQGLPTLQIIIEDGYNKKTVVKKVESLLNKKGSGSVLGEYTYTDTEEIIKSIDKIFDSITYFVAAVAGISLFIAGIGVMNVMYISVAERTEEIAIRRAFGAKSRDIELQFLIESILICVTSGFIGLILGVVFATIIDVLTPDYIKSVVSLSSVIIAVSVSILIGLLFGWIPARAASKKELIDIIK

SEQ ID NO: 33 - protein, 70 aa, Staphylococcus aureus; identical to SEQ ID NO: 27

SEQ ID NO: 34 - protein, 97 aa, Staphylococcus aureus
MAMIKMSPEEIRAKSQSYGQGSDQIRQILSDLTRAQGEIAANWEGQAFSRFEEQFQQLSPKVEKFAQLLEEIKQQLNSTADAVQEQDQQLSNNFGLQ

SEQ ID NO: 35 - DNA, 683 bp, Artificial Sequence
  EsxA-V5-313 fusion
ggtaccgctagccgcgccgccaccatggatgcaatgaagagagggctctgctgtgtgctgctgctgtgtggagcagtcttcgtttcgcccagccaggaaatccatgcccgattcagaagaggatcgaagcttgccatgatcaagatgagccccgaggaaatccgggccaagagccagagctacggccagggcagcgaccagatccggcagatcctgagcgacctgaccagagcccagggcgagatcgccgccaattgggagggccaggccttcagcagattcgaggaacagtttcagcagctgagccccaaggtggagaagttcgcccagctgctggaagagatcaagcagcagctgaacagcaccgccgacgccgtgcaggaacaggatcagcagctgtccaacaacttcgggctgcagggatccgggcccggggcttcaggtaagcctatccctaaccctctcctcggtctcgattctacgcggaccggacctggatcaaagaagcagggcgacgccgacgtgtgtggcgaggtggcctacatccagagcgtggtgtccgactgtcacgtgcccaccgccgagctgagaaccctgctggaaatccggaagctgttcctggaaatccagaaactgaaggtggagctgcagggcctgagcaaagagtgatgagcggccgctcg

SEQ ID NO: 36 - DNA, 1306 bp, Artificial Sequence
  BitC.V5.313
ggtaccgctagccgcgccgccaccatggatgcaatgaagagagggctctgctgtgtgctgctgctgtgtggagcagtcttcgtttcgcccagccaggaaatccatgcccgattcagaagaggatcgaagcttgccaacacccggcacagcgagagcgacaagaacgtgctgaccgtgtacagcccctaccagagcaacctgatccggcccatcctgaacgagttcgagaagcaggaacacgtcaagatcgagatcaagcacggcagcacacaggtgctgctgagcaacctgcacaacgaggacttcagcgagcggggcgacgtgttcatgggcggcgtgctgagcgagacaatcgaccaccccgaggacttcgtgccataccaggacaccagcgtgacccagcagctggaagattaccggtccaacaacaaatacgtgaccagcttcctgctgatgcccaccgtgatcgtggtcaacagcgacctgcagggcgacatcaagatccggggctaccaggacctgctgcagcctatcctgaagggcaagatcgcctacagcaaccccaacaccaccaccaccggctaccagcacatgcgggccatctacagcatgcaccaccgggtgtccgacgtgcaccagttccagaaccacgccatgcagctgagcaagaccagcaaagtgatcgaggacgtggccaagggcaagtactacgccggcctgagctacgagcaggacgcccggacctggaagaacaagggctaccccgtgtccatcgtgtaccccatcgagggcaccatgctgaacgtggacggaatcgccctggtcaagaacgcccacccccaccccaagcggaagaaactggtgcagtacctgaccagcagaagcgtgcagcagcggctggtggccgagttcgacgccaagagcatccggaaggacgtgtccgagcagagcgaccagagcatcgagaacctgaagaacatccccctgatccccaagagcaagctgcccgacatcccccaccacaagtttctggaaatgatccagggatccgggcccggggcttcaggtaagcctatccctaaccctctcctcggtctcgattctacgcggaccggacctggatcaaagaagcagggcgacgccgacgtgtgtggcgaggtggcctacatccagagcgtggtgtccgactgtcacgtgcccaccgccgagctgagaaccctgctggaaatccggaagctgttcctggaaatccagaaactgaaggtggagctgcagggcctgagcaaagagtgatgagcggccgctc

SEQ ID NO: 37 - DNA, 1291 bp, Artificial Sequence
  EsxA.rLuc
gtaccgaattcaccgccgccaacatgaagcttgccatgatcaagatgagccccgaggaaatccgggccaagagccagagctacggccagggcagcgaccagatccggcagatcctgagcgacctgaccagagcccagggcgagatcgccgccaattgggagggccaggccttcagcagattcgaggaacagtttcagcagctgagccccaaggtggagaagttcgcccagctgctggaagagatcaagcagcagctgaacagcaccgccgacgccgtgcaggaacaggatcagcagctgtccaacaacttcgggctgcagggatccgggcccggggccatgggttcgaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatctttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaattctagatttttatagc

SEQ ID NO: 38 - DNA, 1915 bp, Artificial Sequence
  BitC.rLuc
gtaccgaattcaccgccgccaacatgaagcttgccaacacccggcacagcgagagcgacaagaacgtgctgaccgtgtacagcccctaccagagcaacctgatccggcccatcctgaacgagttcgagaagcaggaacacgtcaagatcgagatcaagcacggcagcacacaggtgctgctgagcaacctgcacaacgaggacttcagcgagcggggcgacgtgttcatgggcggcgtgctgagcgagacaatcgaccaccccgaggacttcgtgccataccaggacaccagcgtgacccagcagctggaagattaccggtccaacaacaaatacgtgaccagcttcctgctgatgcccaccgtgatcgtggtcaacagcgacctgcagggcgacatcaagatccggggctaccaggacctgctgcagcctatcctgaagggcaagatcgcctacagcaaccccaacaccaccaccaccggctaccagcacatgcgggccatctacagcatgcaccaccgggtgtccgacgtgcaccagttccagaaccacgccatgcagctgagcaagaccagcaaagtgatcgaggacgtggccaagggcaagtactacgccggcctgagctacgagcaggacgcccggacctggaagaacaagggctaccccgtgtccatcgtgtaccccatcgagggcaccatgctgaacgtggacggaatcgccctggtcaagaacgcccacccccaccccaagcggaagaaactggtgcagtacctgaccagcagaagcgtgcagcagcggctggtggccgagttcgacgccaagagcatccggaaggacgtgtccgagcagagcgaccagagcatcgagaacctgaagaacatccccctgatccccaagagcaagctgcccgacatcccccaccacaagtttctggaaatgatccagggatccgggcccggggccatgggttcgaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatctttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggctgctctggcctttcactacgcctacgagcaccaagacaggatcaaggccatcgtccatatggagagtgtcgtggacgtgatcgagtcctgggacgagtggcctgacatcgaggaggatatcgccctgatcaagagcgaagagggcgagaaaatggtgcttgagaataacttcttcgtcgagaccgtgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagtaattctagatttttatagc

SEQ ID NO: 39 - DNA, 4596 bp, Artificial Sequence
  pMono2.BitC.313
cgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctccaccatgggaaccaattcagtcgagcctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggttaagctcggtaccgctagccgcgccgccaccatggatgcaatgaagagagggctctgctgtgtgctgctgctgtgtggagcagtcttcgtttcgcccagccaggaaatccatgcccgattcagaagaggatcgaagcttgccaacacccggcacagcgagagcgacaagaacgtgctgaccgtgtacagcccctaccagagcaacctgatccggcccatcctgaacgagttcgagaagcaggaacacgtcaagatcgagatcaagcacggcagcacacaggtgctgctgagcaacctgcacaacgaggacttcagcgagcggggcgacgtgttcatgggcggcgtgctgagcgagacaatcgaccaccccgaggacttcgtgccataccaggacaccagcgtgacccagcagctggaagattaccggtccaacaacaaatacgtgaccagcttcctgctgatgcccaccgtgatcgtggtcaacagcgacctgcagggcgacatcaagatccggggctaccaggacctgctgcagcctatcctgaagggcaagatcgcctacagcaaccccaacaccaccaccaccggctaccagcacatgcgggccatctacagcatgcaccaccgggtgtccgacgtgcaccagttccagaaccacgccatgcagctgagcaagaccagcaaagtgatcgaggacgtggccaagggcaagtactacgccggcctgagctacgagcaggacgcccggacctggaagaacaagggctaccccgtgtccatcgtgtaccccatcgagggcaccatgctgaacgtggacggaatcgccctggtcaagaacgcccacccccaccccaagcggaagaaactggtgcagtacctgaccagcagaagcgtgcagcagcggctggtggccgagttcgacgccaagagcatccggaaggacgtgtccgagcagagcgaccagagcatcgagaacctgaagaacatccccctgatccccaagagcaagctgcccgacatcccccaccacaagtttctggaaatgatccagggatccgggcccggggcttcaggtaagcctatccctaaccctctcctcggtctcgattctacgcggaccggacctggatcaaagaagcagggcgacgccgacgtgtgtggcgaggtggcctacatccagagcgtggtgtccgactgtcacgtgcccaccgccgagctgagaaccctgctggaaatccggaagctgttcctggaaatccagaaactgaaggtggagctgcagggcctgagcaaagagtgatgagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagttggcattataagaaagcattgcttatcaatttgttgcaacgaacaggtcactatcagtcaaaataaaatcattatttgccatccagctgcagctctggcc

SEQ ID NO: 40 - DNA, 6763 bp, Artificial Sequence
  pMVA.BitC.313
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcatctggaaacgggcatctccatttaagactagatgccacggggtttaaaatactaatcatgacattttgtagagcgtaattacttagtaaatccgccgtactaggttcatttcctcctcgtttggatctcacatcagaaattaaaataatcttagaaggatgcagttgttttttgatggatcgtagatattcctcatcaacgaaccgagtcactagagtcacatcacgcaatccatttaaaataggatcatgatggcggccgtcaattagcatccatttgatgatcactcctaaattatagaaatgatctctcaaataacgtatatgtgtaccgggagcagatcctatatacactacggtggcaccatctaatataccgtgtcgctgtaacttactaagaaaaaataattctcctagtaatagttttaactgtccttgatacggtagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattatgagtcgatgtaacactttctacacaccgattgatacatatcattacctcctattatttctatctcggtttcctcacccaatcgtttagaaaaggaagcctccttaaagcatttcatacacacagcagttagttttaccaccatttcagataatggaataagattcaaaatattattaaacggtttacgttgaaatgtcccatcgagtgcggctactataactatttttccttcgtttgccatacagatcctacgtactcgagataaaaagcggccttggccgaggcggccacgcgtgcggccgctcatcactctttgctcaggccctgcagctccaccttcagtttctggatttccaggaacagcttccggatttccagcagggttctcagctcggcggtgggcacgtgacagtcggacaccacgctctggatgtaggccacctcgccacacacgtcggcgtcgccctgcttctttgatccaggtccggtccgcgtagaatcgagaccgaggagagggttagggataggcttacctgaagccccgggcccggatccctggatcatttccagaaacttgtggtgggggatgtcgggcagcttgctcttggggatcagggggatgttcttcaggttctcgatgctctggtcgctctgctcggacacgtccttccggatgctcttggcgtcgaactcggccaccagccgctgctgcacgcttctgctggtcaggtactgcaccagtttcttccgcttggggtgggggtgggcgttcttgaccagggcgattccgtccacgttcagcatggtgccctcgatggggtacacgatggacacggggtagcccttgttcttccaggtccgggcgtcctgctcgtagctcaggccggcgtagtacttgcccttggccacgtcctcgatcactttgctggtcttgctcagctgcatggcgtggttctggaactggtgcacgtcggacacccggtggtgcatgctgtagatggcccgcatgtgctggtagccggtggtggtggtgttggggttgctgtaggcgatcttgcccttcaggataggctgcagcaggtcctggtagccccggatcttgatgtcgccctgcaggtcgctgttgaccacgatcacggtgggcatcagcaggaagctggtcacgtatttgttgttggaccggtaatcttccagctgctgggtcacgctggtgtcctggtatggcacgaagtcctcggggtggtcgattgtctcgctcagcacgccgcccatgaacacgtcgccccgctcgctgaagtcctcgttgtgcaggttgctcagcagcacctgtgtgctgccgtgcttgatctcgatcttgacgtgttcctgcttctcgaactcgttcaggatgggccggatcaggttgctctggtaggggctgtacacggtcagcacgttcttgtcgctctcgctgtgccgggtgttggcaagcttcgatcctcttctgaatcgggcatggatttcctggctgggcgaaacgaagactgctccacacagcagcagcacacagcagagccctctcttcattgcatccatggtggcggcgcggctagcggtaccttatttatattccaaaaaaaaaaaataaaatttcaatttttacaaaaagaattcatccactttggataagaaatctgcatgataaatatattgatatcctaccacctattaaagtaccattatctaatagcaataagatagataaacaaatgttttttgatgaagttattacgtggataaatatatatcttcaggaaaagggtattatgttaccagatgatataagagaactcagagatgctattattccttaactagttacgtctctttaggtacttattttgatacgttacaagtaaaaaactatcaaatataaatggaatctgattctaatatagcgattgaagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccggccgcgaagttcctatactttctagagaataggaacttcaacaatgtctggaaagaactgtccttcatcgatacctatcacggagaaatctgtaattgattccaagacatcacatagtttagttgcttccaatgcttcaaaattattcttatcatgcgtccatagtcccgttccgtatctattatcgttagaatattttatagtcacgcatttatattgagctatttgataacgtctaactcgtctaattaattctgtacttttacctgaaaacatggggccgattatcaactgaatatgtccgccgttcatgatgacaataaagaattaattattgttcactttattcgactttaatatatccatcacgttagaaaatgcgatatcgcgacgaggatctatgtatctaacaggatctattgcggtggtagctagagctgattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttatgagctcgcgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

(19) United States
(12) Patent Application Publication
(10) Pub. No.: US 2015/0259388 A1
(43) Pub. Date: Sep. 17, 2015

(54) STAPHYLOCOCCUS AUREUS ANTIGENS

(71) Applicant: ISIS INNOVATION LIMITED, Oxford, Oxfordshire (GB)

(72) Inventors: David Wyllie, Oxford (GB); Christine Rollier, Oxford (GB); Yuko Yamaguchi, Oxford (GB); Pauline Van Diemen, Oxford (GB); Adrian Hill, Oxford (GB)

(21) Appl. No.: 14/433,565

(22) PCT Filed: Oct. 7, 2013
(86) PCT No.: PCT/GB2013/052607

§ 371 Date: Apr. 3, 2015

(30) Foreign Application Priority Data
Oct. 5, 2012 (GB) 1217868.7

(51) Int. Cl. C07K 14/31; C12N 7/00; A61K 39/085

(52) U.S. Cl. CPC C07K 14/31; A61K 39/085; C12N 7/00; C12N 2710/24042; C12N 2710/10042; A61K 39/00

(57) ABSTRACT

The present invention provides a non-replicating poxvirus vector comprising a nucleic acid sequence encoding a Staphylococcus aureus antigen, wherein the nucleic acid sequence encoding a Staphylococcus aureus antigen comprises a nucleic acid sequence having at least 70% sequence identity to a nucleic acid sequence selected from SEQ ID NOs: 1, 2, 3, 4, 5, 6, 7, 8, 9, 10, 11, 12, 13, 14, 15, and 16. The present invention also provides compositions and uses of the vectors in methods of medical treatment.

This patent application claims priority to GB 1217868.7 filed on 5 Oct. 2012, which is hereby incorporated by reference in its entirety.

The present invention relates to Staphylococcus aureus antigens, viral vectors comprising nucleic acid sequences encoding Staphylococcus aureus antigens, and their use as immunogenic compositions.

Staphylococcus aureus (S. aureus) is one of the most important bacterial pathogens of man, causing skin, wound, and deep infections. It also causes a range of diseases in livestock, notably bovine mastitis. Morbidity and mortality are associated with invasion of tissues and abscess formation, and treatment of these conditions commonly requires surgery and prolonged antibiotic therapy with compounds such as flucloxacillin, vancomycin, teicoplanin, and linezolid.

There is an ongoing human and financial impact of nosocomial S. aureus disease within the United Kingdom and elsewhere. Internationally, the organism is responsible for about half the cost of health care-associated infection. S. aureus is a highly clonal organism, and a relatively limited number of successful (and frequently antibiotic-resistant) strains (such as methicillin-resistant S. aureus or MRSA) contribute disproportionately to the burden of disease. For example, an epidemic of hospital based MRSA is ongoing in Europe, while an epidemic of community disease, due to methicillin-resistant S. aureus clones such as USA300, exists in North America. There is also an emerging zoonotic threat from pig strains.

S. aureus carriage, a state in which the organism can be cultured from the nares without evident clinical impact, is common in both humans (approximately 30% frequency) and some animals, particularly pigs. Long-term carriage of S. aureus is required for organism spread in some settings. Carriage raises risk of invasive disease, and risk can be decreased by drug-based decolonisation in some groups. This decolonisation is usually transient, and is heavily reliant on chlorhexidine and mupiricin, resistance to both of which is increasing.

Increasingly, it is recognised that both S. aureus carriage and invasive disease are characterised by the presence of the bacteria in an intracellular state, with both epithelial cells and neutrophils being infected.

There are at present no effective, commercially available vaccines against S. aureus. A recent failure involved Merck's V710 trial of the protein IsdB in prevention of wound infection. Whilst some boosting of antibody responses was reported, it was recently announced that there was no efficacy in a Phase III study. Passive immunization with anti-capsular antibodies (AltaStaph), anti-ClfA and SdrG (Veronate), and an anti-lipoteichoic acid antibody (Pagibaximab) also failed in clinical trials.

There is therefore a need for new immunogenic compositions that demonstrate improved immunogenicity when used in the prevention and treatment of S. aureus infections, in particular in human subjects. In particular, there is a need for new immunogenic compositions that can produce an improved antigen-specific T cell response, as well as an improved antibody response.

The present invention addresses one or more of the above problems by providing viral vectors encoding S. aureus antigens, together with corresponding compositions and uses of said vectors and compositions in the prevention and treatment of S. aureus infections and carriage states. The present invention also provides S. aureus polypeptide antigen compositions for use in the prevention and treatment of S. aureus infections and carriage states.

The viral vectors and compositions of the invention enable an immune response against S. aureus to be stimulated in an individual, and provide improved immunogenicity and efficacy.

In one aspect, the invention provides a non-replicating poxvirus vector comprising a nucleic acid sequence encoding a Staphylococcus aureus antigen, wherein the nucleic acid sequence encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to a nucleic acid sequence selected from SEQ ID NOs: 1, 2, 3, 4, 5, 6, 7, 8, 9, 10, 11, 12, 13, 14, 15, and 16.

In another aspect, the invention provides an adenovirus vector comprising a nucleic acid sequence encoding a Staphylococcus aureus antigen, wherein the nucleic acid sequence encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to a nucleic acid sequence selected from SEQ ID NOs: 1, 2, 3, 4, 5, 6, 7, 8, 9, 10, 11, 12, 13, 14, 15, and 16.

In a related aspect, the invention provides a non-replicating poxvirus vector comprising a nucleic acid sequence encoding a Staphylococcus aureus antigen, wherein the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33.

In another related aspect, the invention provides an adenovirus vector comprising a nucleic acid sequence encoding a Staphylococcus aureus antigen, wherein the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33.

The present inventors have found that the S. aureus antigens encoded by the nucleic acid sequences of SEQ ID NOs: 1-16 (and which nucleic acid sequences encode the corresponding amino acid sequences of SEQ ID NOs: 18-33, respectively) can be used to generate effective immune responses in individuals against S. aureus. In particular, the inventors have found that a highly effective immune response against S. aureus is obtained when one or more members of this group of antigens is delivered to the subject using a viral vector, such as a non-replicating poxvirus vector or an adenovirus vector.

Non-replicating poxviruses and adenoviruses represent groups of viruses which may be used as vectors for the delivery of genetic material into a target cell. Viral vectors serve as antigen delivery vehicles and also have the power to activate the innate immune system through binding cell surface molecules that recognise viral elements. A recombinant viral vector can be produced that carries nucleic acid encoding a given antigen. The viral vector can then be used to deliver the nucleic acid to a target cell, where the encoded antigen is produced by the target cell's own molecular machinery. As “non-self”, the produced antigen generates an immune response in the target subject.

Without wishing to be bound by any one particular theory, the inventors believe that antigen delivery using the viral vectors of the invention stimulates, amongst other responses, a T cell response in the subject. Thus, the inventors believe that one way in which the present invention provides for protection against S. aureus infection is by stimulating T cell responses and the cell-mediated immunity system. In addition, humoral (antibody) based protection can also be achieved.

The viral vector of the invention may be a non-replicating poxvirus vector. As used herein, a non-replicating (or replication-deficient) viral vector is a viral vector which lacks the ability to productively replicate following infection of a target cell. Thus, a non-replicating viral vector cannot produce copies of itself following infection of a target cell. Non-replicating viral vectors may therefore advantageously have an improved safety profile as compared to replication-competent viral vectors.

In one embodiment, the non-replicating poxvirus vector is selected from: a Modified Vaccinia virus Ankara (MVA) vector, a NYVAC vaccinia virus vector, a canarypox (ALVAC) vector, and a fowlpox (FPV) vector. MVA and NYVAC are both attenuated derivatives of vaccinia virus. Compared to vaccinia virus, MVA lacks approximately 26 of the approximately 200 open reading frames.

In one embodiment, the non-replicating poxvirus vector is an MVA vector.

The viral vector of the invention may be an adenovirus vector. In one embodiment, the adenovirus vector is a non-replicating adenovirus vector (wherein non-replicating is defined as above). Adenoviruses can be rendered non-replicating by deletion of the E1 or both the E1 and E3 gene regions. Alternatively, an adenovirus may be rendered non-replicating by alteration of the E1 or of the E1 and E3 gene regions such that said gene regions are rendered non-functional. For example, a non-replicating adenovirus may lack a functional E1 region or may lack functional E1 and E3 gene regions. In this way the adenoviruses are rendered replication incompetent in most mammalian cell lines and do not replicate in immunised mammals. Most preferably, both E1 and E3 gene region deletions are present in the adenovirus, thus allowing a greater size of transgene to be inserted. This is particularly important to allow larger antigens to be expressed, or when multiple antigens are to be expressed in a single vector, or when a large promoter sequence, such as the CMV promoter, is used. Deletion of the E3 as well as the E1 region is particularly favoured for recombinant Ad5 vectors. Optionally, the E4 region can also be engineered.

In one embodiment, the adenovirus vector is selected from: a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

In a preferred embodiment, the nucleic acid sequence encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to the nucleic acid sequence of SEQ ID NO: 1.

Thus, in one embodiment, the nucleic acid sequence encoding a Staphylococcus aureus antigen encodes a Staphylococcus aureus BitC polypeptide, and the Staphylococcus aureus antigen is therefore a Staphylococcus aureus BitC polypeptide.

The present inventors have discovered that an S. aureus BitC polypeptide is particularly suitable as an antigen in the present invention. The BitC polypeptide has not been previously recognised as protective against S. aureus. The BitC gene is believed to undergo a significant increase in expression in the first six hours following internalisation of S. aureus in an infected cell. Based on primary sequence analysis, the BitC polypeptide is predicted to be a 322 amino acid lipoprotein, which may be attached to the outside of the cell, and homologous to bacterial ABC transporters, a class of proteins which regulate import and export from bacterial cells.

In one embodiment, the nucleic acid sequence encoding a Staphylococcus aureus antigen comprises (or consists of) a nucleic acid sequence encoding a polypeptide comprising (or consisting of) an amino acid sequence having at least 70% (such as at least 70, 75, 80, 82, 84, 86, 88, 90, 92, 94, 96, 98, 99 or 100%) sequence identity to an amino acid sequence selected from any one of the following sequences (using NCBI reference identifiers): YP_{—}042322, YP_{—}005740974.1, YP_{—}005748938.1, NP_{—}370749.1, YP_{—}003281141.1, YP_{—}001440814.1, YP_{—}001315364.1, YP_{—}001245592.1, NP_{—}373461.1, EJU84038.1, BAB41439.1, BAF77107.1, ACY10135.1, ADC36436.1, EJE57192.1, ABR51077.1, ABQ48016.1, EIK36045.1, EIK27679.1, EIK27108.1, EIK26538.1, EIK35558.1, EIK34570.1, EIK19196.1, EIK16248.1, EIK10998.1, EIK09850.1, EIK09612.1, EIK17389.1, EID41792.1, EHT91838.1, EHT83400.1, EHT96369.1, EHT48254.1, EHT41598.1, EHT57733.1, EHT52681.1, EHT41283.1, EHT22180.1, EHT24561.1, EHS28916.1, EHS15156.1, EHS10062.1, EHS16169.1, EH099330.1, EHM67629.1, CBX33588.1, EGS94503.1, EGL91665.1, EGG62099.1, EFT86188.1, ZP_{—}06857433.1, ZP_{—}05143621.2, ZP_{—}04838635.1, ZP_{—}06928711.1, ZP_{—}06815304.1, ZP_{—}06333964.1, ZP_{—}06301614.1, ZP_{—}05703732.1, ZP_{—}05696656.1, ZP_{—}05692538.1, ZP_{—}05695235.1, ZP_{—}05690060.1, ZP_{—}05684148.1, ZP_{—}05680915.1, ZP_{—}05642942.1, ZP_{—}04864734.1, EFH37595.1, EFG45588.1, EFC04194.1, EFB96732.1, EEV84762.1, EEV81062.1, EEV65098.1, EEV77051.1, EEV74411.1, EEV71841.1, EEV67067.1, EEV26275.1, EES94438.1, BAB56387.1.

The viral vector of the invention, as described above, can be used to deliver a single antigen to a target cell. Advantageously, the viral vector of the invention can also be used to deliver multiple (different) antigens to a target cell.

Thus, in one embodiment, the vector further comprises at least one additional nucleic acid sequence encoding a Staphylococcus aureus antigen. The vector may therefore comprise a first nucleic acid sequence (as described above) and an additional (e.g. a second) nucleic acid sequence. The antigen so encoded can be different from the antigen encoded by the first nucleic acid sequence.

In one embodiment, the at least one additional nucleic acid sequence comprises (or consists of) a nucleic acid sequence having at least 70% (such as at least 70, 75, 80, 82, 84, 86, 88, 90, 92, 94, 96, 98, 99 or 100%) sequence identity to a nucleic acid sequence selected from SEQ ID NOs: 1, 2, 3, 4, 5, 6, 7, 8, 9, 10, 11, 12, 13, 14, 15, 16, and 17.

In one embodiment, the at least one additional nucleic acid sequence comprises (or consists of) a nucleic acid sequence having at least 70% (such as at least 70, 75, 80, 82, 84, 86, 88, 90, 92, 94, 96, 98, 99 or 100%) sequence identity to the nucleic acid sequence of SEQ ID NO: 17.

Thus, in one embodiment, the at least one additional nucleic acid sequence encodes an S. aureus EsxA polypeptide.

The nucleic acid sequences as described above may comprise a nucleic acid sequence encoding a Staphylococcus aureus antigen, wherein said antigen comprises a fusion protein. The fusion protein may comprise a Staphylococcus aureus antigen polypeptide fused to one or more further polypeptides, for example an epitope tag, another antigen, or a protein that increases immunogenicity (e.g. a flagellin).

In one embodiment, the vector (as described above) further comprises a nucleic acid sequence encoding an adjuvant (for example, a cholera toxin, an E. coli lethal toxin, or a flagellin).

In another aspect, the invention provides a nucleic acid sequence encoding a vector, as described above. Thus, the nucleic acid sequence may encode a non-replicating poxvirus vector as described above. Alternatively, the nucleic acid sequence may encode an adenovirus vector as described above.

The nucleic acid sequence encoding a vector (as described above) may be generated by the use of any technique for manipulating and generating recombinant nucleic acid known in the art.

In one aspect, the invention provides a method of making a vector (as described above), comprising providing a nucleic acid, wherein the nucleic acid comprises a nucleic acid sequence encoding a vector (as described above); transfecting a host cell with the nucleic acid; culturing the host cell under conditions suitable for the propagation of the vector; and obtaining the vector from the host cell.

As used herein, “transfecting” may mean any non-viral method of introducing nucleic acid into a cell. The nucleic acid may be any nucleic acid suitable for transfecting a host cell. Thus, in one embodiment, the nucleic acid is a plasmid. The host cell may be any cell in which a vector (i.e. a non-replicating poxvirus vector or an adenovirus vector, as described above) may be grown. As used herein, “culturing the host cell under conditions suitable for the propagation of the vector” means using any cell culture conditions and techniques known in the art which are suitable for the chosen host cell, and which enable the vector to be produced in the host cell. As used herein, “obtaining the vector”, means using any technique known in the art that is suitable for separating the vector from the host cell. Thus, the host cells may be lysed to release the vector. The vector may subsequently be isolated and purified using any suitable method or methods known in the art.

In one aspect, the invention provides a host cell comprising a nucleic acid sequence encoding a vector, as described above. The host cell may be any cell in a which a vector (i.e. a non-replicating poxvirus vector or an adenovirus vector, as described above) may be grown or propagated. The host cell may be selected from: a 293 cell (also known as a HEK, or human embryonic kidney, cell), a CHO cell (Chinese Hamster Ovary), a CCL81.1 cell, a Vero cell, a HELA cell, a Per.C6 cell, a BHK cell (Baby Hamster Kidney), a primary CEF cell (Chicken Embryo Fibroblast), a duck embryo fibroblast cell, or a DF-1 cell.

In another aspect, the invention provides an isolated Staphylococcus aureus polypeptide antigen, wherein the polypeptide antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33. In a preferred embodiment, the polypeptide antigen comprises (or consists of) an amino acid sequence having at least 70% (such as at least 70, 75, 80, 82, 84, 86, 88, 90, 92, 94, 96, 98, 99 or 100%) sequence identity to SEQ ID NO: 18. Thus, in one embodiment, the polypeptide antigen is a Staphyloccus aureus BitC polypeptide.

The present invention also provides compositions comprising vectors as described above.

In one aspect, the invention provides a composition comprising a vector (as described above) and a pharmaceutically-acceptable carrier.

Substances suitable for use as pharmaceutically-acceptable carriers are known in the art. Non-limiting examples of pharmaceutically-acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage. Examples of buffering agents include, but are not limited to, sodium succinate (pH 6.5), and phosphate buffered saline (PBS; pH 7.4).

In addition to a pharmaceutically-acceptable carrier, the composition of the invention can be further combined with one or more of a salt, excipient, diluent, adjuvant, immunoregulatory agent and/or antimicrobial compound.

The composition may be formulated as a neutral or salt form. Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the composition (as described above) further comprises a second viral vector, wherein the second viral vector comprises a nucleic acid sequence encoding a Staphylococcus aureus antigen. By way of example, the Staphylococcus aureus antigen may be an antigen as described above.

In one embodiment, the second viral vector is a vector as described above. Thus, in one embodiment, the second viral vector is selected from a non-replicating poxvirus vector and an adenovirus vector.

In one embodiment, the first and second viral vectors are provided separately.

In one embodiment, the first and second viral vectors encode different antigens. Thus, in one embodiment, the second vector comprises a nucleic acid sequence encoding an antigen that is different from the antigen encoded by the first vector. Thus, in one embodiment, a composition of the invention can be used to deliver to a subject, and so stimulate an immune response against, two different antigens.

In one embodiment, the nucleic acid sequence (of the second vector) encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to the nucleic acid sequence of SEQ ID NO: 17.

Thus, in one embodiment, the nucleic acid sequence (of the second vector) encoding a Staphylococcus aureus antigen encodes an S. aureus EsxA polypeptide, and the Staphylococcus aureus antigen is therefore an S. aureus EsxA polypeptide.

In one embodiment, the second viral vector is an adenovirus vector (for example an adenovirus vector as described above).

In one embodiment, the second viral vector is a non-replicating poxvirus vector. In one embodiment, the second vector is a non-replicating poxvirus vector selected from: a Modified Vaccinia virus Ankara (MVA) vector, a NYVAC vaccinia virus vector, a canarypox (ALVAC) vector, and a fowlpox (FPV) vector. In one embodiment, the second viral vector is an MVA vector.

In one embodiment, the composition (as described above) further comprises at least one Staphylococcus aureus polypeptide antigen (i.e. an antigen present in the composition in the form of a polypeptide). Thus, the composition may comprise both viral vector and polypeptide. In one embodiment, the presence of a polypeptide antigen means that, following administration of the composition to a subject, an improved simultaneous T cell and antibody response can be achieved. In one embodiment, the T cell and antibody response achieved surpasses that achieved when either a viral vector or a polypeptide antigen is used alone.

In one embodiment, the polypeptide antigen is not bonded to the viral vector. In one embodiment, the polypeptide antigen is a separate component to the viral vector. In one embodiment, the polypeptide antigen is provided separately from the viral vector.

In one embodiment, the polypeptide antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from: SEQ ID NOs: 18, 19, 20, 21, 22, 23, 24, 25, 26, 27, 28, 29, 30, 31, 32, 33, and 34.

In one embodiment, the polypeptide antigen is a variant of the antigen encoded by the viral vector. In one embodiment, the polypeptide antigen is a fragment of the antigen encoded by the viral vector. In one embodiment, the polypeptide antigen comprises at least part of a polypeptide sequence encoded by a nucleic acid sequence of the vector. Thus, the polypeptide antigen may correspond to at least part of the antigen encoded by the viral vector.

The polypeptide antigen may be the same as (or similar to) that encoded by a nucleic acid sequence of the viral vector of the composition. Thus, administration of the composition comprising a viral vector and a polypeptide antigen may be used to achieve an enhanced immune response against a single antigen, wherein said enhanced immune response comprises a combined T cell and an antibody response, as described above.

In another aspect, the invention provides a composition comprising a Staphylococcus aureus polypeptide antigen and a pharmaceutically acceptable carrier, wherein the polypeptide antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33. In a preferred embodiment, the polypeptide antigen comprises (or consists of) an amino acid sequence having at least 70% (such as at least 70, 75, 80, 82, 84, 86, 88, 90, 92, 94, 96, 98, 99 or 100%) sequence identity to SEQ ID NO: 18. Thus, in one embodiment, the polypeptide antigen is a Staphyloccus aureus BitC polypeptide. In one embodiment, the composition further comprises a second Staphylococcus aureus polypeptide antigen, wherein said second antigen is a Staphylococcus aureus polypeptide antigen as described above.

It is recognised that bacteria may be disadvantaged when cell-surface antigens are targeted by antibodies, which can inhibit bacterial survival by disrupting critical protein functions or by recruitment of complement and phagocytes. Thus, in another aspect, the invention provides a composition comprising a monoclonal antibody and a pharmaceutically acceptable carrier, wherein the monoclonal antibody is an antibody against a polypeptide comprising an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33. In one embodiment, the monoclonal antibody is an antibody against a polypeptide comprising an amino acid sequence having at least 70% (such as at least 70, 75, 80, 82, 84, 86, 88, 90, 92, 94, 96, 98, 99 or 100%) sequence identity to SEQ ID NO: 18. Thus, in one embodiment, the monoclonal antibody (which may be a humanised monoclonal antibody) is an anti-BitC antibody.

In one embodiment, a composition of the invention (as described above) further comprises an adjuvant. Non-limiting examples of adjuvants suitable for use with compositions of the present invention include aluminium phosphate, aluminium hydroxide, and related compounds; monophosphoryl lipid A, and related compounds; outer membrane vesicles from bacteria; oil-in-water emulsions such as MF59; liposomal adjuvants, such as virosomes, Freund's adjuvant and related mixtures; poly-lactid-co-glycolid acid (PLGA) particles; cholera toxin; E. coli lethal toxin; and flagellin.

The compositions (as described above) can be employed as vaccines. Thus, a composition of the invention may be a vaccine composition.

As used herein, a vaccine is a formulation that, when administered to an animal subject such as a mammal (e.g. a human, bovine, porcine, ovine, caprine, equine, cervine, canine or feline subject; in particular a human subject), stimulates a protective immune response against an infectious disease. The immune response may be a humoral and/or a cell-mediated immune response. Thus, the vaccine may stimulate B cells and/or T cells.

The term “vaccine” is herein used interchangeably with the terms “therapeutic/prophylactic composition”, “immunogenic composition”, “formulation”, “antigenic composition”, or “medicament”.

In one aspect, the invention provides a vector (as described above) or a composition (as described above) for use in medicine.

In one aspect, the invention provides a non-replicating poxvirus vector for use in a method of inducing a T cell response to a Staphylococcus aureus antigen in a subject. The present inventors have discovered that non-replicating poxvirus vectors are particularly suitable for inducing T cell responses in a subject against S. aureus. Thus, a non-replicating poxvirus vector can be used to stimulate a protective immune response via the cell-mediated immune system.

In one embodiment, the T cell is a T helper cell (Tₕ cell). In one embodiment, the T cell is a Tₕ17 cell.

In one embodiment, the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, 33, and 34.

In one embodiment, the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to an amino acid sequence selected from SEQ ID NOs: 18, 19, 20, 21, 22, 23, 24, 25, 26, 27, 28, 29, 30, 31, 32, and 33.

In one embodiment, the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to SEQ ID NO: 18.

In one embodiment, the Staphylococcus aureus antigen is a Staphylococcus aureus BitC polypeptide.

In one embodiment, the Staphylococcus aureus antigen comprises (or consists of) an amino acid sequence having at least 70% (such as at least 70, 75, 80, 82, 84, 86, 88, 90, 92, 94, 96, 98, 99 or 100%) sequence identity to SEQ ID NO: 34.

In one embodiment, the Staphylococcus aureus antigen is a Staphylococcus aureus EsxA polypeptide.

In one embodiment, the method of inducing a T cell response (as described above) comprises administering to a subject an effective amount of a non-replicating poxvirus vector comprising a nucleic acid encoding a Staphylococcus aureus antigen (for example an antigen as described above).

In one embodiment, the nucleic acid encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to a nucleic acid sequence selected from SEQ ID NOs: 1, 2, 3, 4, 5, 6, 7, 8, 9, 10, 11, 12, 13, 14, 15, 16, and 17.

In one embodiment, the nucleic acid encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to a nucleic acid sequence selected from SEQ ID NOs: 1, 2, 3, 4, 5, 6, 7, 8, 9, 10, 11, 12, 13, 14, 15, and 16.

In one embodiment, the nucleic acid sequence encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to the nucleic acid sequence of SEQ ID NO: 1.

In one embodiment, the nucleic acid sequence encoding a Staphylococcus aureus antigen encodes a Staphylococcus aureus BitC polypeptide, and the Staphylococcus aureus antigen is therefore a Staphylococcus aureus BitC polypeptide.

In one embodiment, the nucleic acid encoding a Staphylococcus aureus antigen comprises (or consists of) a nucleic acid sequence having at least 70% (such as at least 70, 75, 80, 82, 84, 86, 88, 90, 92, 94, 96, 98, 99 or 100%) sequence identity to the nucleic acid sequence of SEQ ID NO: 17.

Thus, in one embodiment, the nucleic acid encoding a Staphylococcus aureus antigen encodes an S. aureus EsxA polypeptide, and the Staphylococcus aureus antigen is therefore an S. aureus EsxA polypeptide.

In a related aspect, the present invention provides a vector, as described above, for use in inducing a T cell response to a Staphylococcus aureus antigen in a subject. In one embodiment, the T cell is a T helper cell (Tₕ cell). In one embodiment, the T cell is a Tₕ17 cell.

In one aspect, the invention provides a vector (as described above) or a composition (as described above) for use in a method of inducing an immune response in a subject. The immune response may be against a Staphylococcus aureus antigen and/or infection. Thus, the vectors and compositions of the invention can be used to induce an immune response in a subject against a Staphylococcus aureus antigen (for example, as immunogenic compositions).

In one aspect, the invention provides a vector (as described above) or a composition (as described above) for use in a method of reducing Staphylococcus aureus carriage in a subject. As discussed above, S. aureus carriage is a highly important factor in S. aureus transmission and increases the risk of development of invasive disease. The vectors and compositions of the invention can be used to treat individuals carrying S. aureus, such that the number of S. aureus bacteria present on or in the individual is reduced (for example, by 50, 60, 70, 80 or 90%, as compared to prior to treatment) or effectively eliminated (for example, by reducing the number of S. aureus bacteria present on or in the individual by greater than 99%, such as 99.5 or 99.9 or 99.99%, as compared to prior to treatment).

In one aspect, the invention provides a vector (as described above) or a composition (as described above) for use in a method of preventing or treating a Staphylococcus aureus infection in a subject.

As used herein, the term “preventing” includes preventing the initiation of Staphylococcus aureus infection and/or reducing the severity of intensity of a Staphylococcus aureus infection. Thus, “preventing” encompasses vaccination.

As used herein, the term “treating” embraces therapeutic and preventative/prophylactic measures (including post-exposure prophylaxis) and includes post-infection therapy and amelioration of a Staphylococcus aureus infection.

Each of the above-described methods can comprise the step of administering to a subject an effective amount, such as a therapeutically effective amount, of a vector or a compound of the invention.

In this regard, as used herein, an effective amount is a dosage or amount that is sufficient to achieve a desired biological outcome. As used herein, a therapeutically effective amount is an amount which is effective, upon single or multiple dose administration to a subject (such as a mammalian subject, in particular a human subject) for treating, preventing, curing, delaying, reducing the severity of, ameliorating at least one symptom of a disorder or recurring disorder, or prolonging the survival of the subject beyond that expected in the absence of such treatment.

Accordingly, the quantity of active ingredient to be administered depends on the subject to be treated, capacity of the subject's immune system to generate a protective immune response, and the degree of protection required. Precise amounts of active ingredient required to be administered may depend on the judgement of the practitioner and may be particular to each subject.

Administration to the subject can comprise administering to the subject a vector (as described above) or a composition (as described above) wherein the composition is sequentially administered multiple times (for example, wherein the composition is administered two, three or four times). Thus, in one embodiment, the subject is administered a vector (as described above) or a composition (as described above) and is then administered the same vector or composition (or a substantially similar vector or composition) again at a different time.

In one embodiment, administration to a subject comprises administering a vector (as described above) or a composition (as described above) to a subject, wherein said composition is administered substantially prior to, simultaneously with, or subsequent to, another immunogenic composition.

Prior, simultaneous and sequential administration regimes are discussed in more detail below.

In certain embodiments, the above-described methods further comprise the administration to the subject of a second viral vector, wherein the second viral vector comprises a nucleic acid sequence encoding a Staphylococcus aureus antigen. Preferably, the second viral vector is a viral vector of the invention as described above (i.e. a non-replicating poxvirus vector or an adenovirus vector as described above).

In one embodiment, the first and second vectors encode the same antigen. In one embodiment, the first and second vectors encode different antigens.

In one embodiment, the first vector is an adenovirus vector (as described above) and the second vector is a non-replicating poxvirus vector (as described above).

In one embodiment, the first and second vectors are administered sequentially, in any order. Thus, the first (“1”) and second (“2”) vectors may be administered to a subject in the order 1-2, or in the order 2-1.

As used herein, “administered sequentially” has the meaning of “sequential administration”, as defined below. Thus, the first and second vectors are administered at (substantially) different times, one after the other.

In one embodiment, the first and second vectors are administered as part of a prime-boost administration protocol. Thus, the first vector may be administered to a subject as the “prime” and the second vector subsequently administered to the same subject as the “boost”.

In one embodiment, the first vector is an adenovirus vector prime, and the second vector is a non-replicating poxvirus vector boost.

In one embodiment, each of the above-described methods further comprises the step of administration to the subject of a Staphylococcus aureus polypeptide antigen. In one embodiment, the Staphylococcus aureus polypeptide antigen is a Staphylococcus aureus polypeptide antigen as described above.

In one embodiment, the polypeptide antigen is administered separately from the administration of a viral vector; preferably the polypeptide antigen and a viral vector are administered sequentially, in any order. Thus, in one embodiment, the viral vector (“V”) and the polypeptide antigen (“P”) may be administered in the order V-P, or in the order P-V.

As used herein, the term polypeptide embraces peptides and proteins.

In certain embodiments, the above-described methods further comprise the administration to the subject of an adjuvant. Adjuvant may be administered with one, two, or all three of: a first vector, a second vector, and a polypeptide antigen.

The immunogenic compositions, therapeutic formulations, medicaments, pharmaceutical compositions, and prophylactic formulations (e.g. vaccines) of the invention may be given in a single dose schedule (i.e. the full dose is given at substantially one time). Alternatively, the immunogenic compositions, therapeutic formulations, medicaments, pharmaceutical compositions, and prophylactic formulations (e.g. vaccines) of the invention may be given in a multiple dose schedule.

A multiple dose schedule is one in which a primary course of treatment (e.g. vaccination) may be with 1-6 separate doses, followed by other doses given at subsequent time intervals required to maintain and or reinforce the immune response, for example (for human subjects), at 1-4 months for a second dose, and if needed, a subsequent dose(s) after a further 1-4 months.

The dosage regimen will be determined, at least in part, by the need of the individual and be dependent upon the judgment of the practitioner (e.g. doctor or veterinarian).

Simultaneous administration means administration at (substantially) the same time.

Sequential administration of two or more compositions/therapeutic agents/vaccines means that the compositions/therapeutic agents/vaccines are administered at (substantially) different times, one after the other.

For example, sequential administration may encompass administration of two or more compositions/therapeutic agents/vaccines at different times, wherein the different times are separated by a number of days (for example, 1, 2, 5, 10, 15, 20, 30, 60, 90, 100, 150 or 200 days).

For example, in one embodiment, the vaccine of the present invention may be administered as part of a ‘prime-boost’ vaccination regime.

In one embodiment, the immunogenic compositions, therapeutic formulations, medicaments, pharmaceutical compositions, and prophylactic formulations (e.g. vaccines) of the invention can be administered to a subject such as a mammal (e.g. a human, bovine, porcine, ovine, caprine, equine, cervine, canine or feline subject) in conjunction with (simultaneously or sequentially) one or more immunoregulatory agents selected from, for example, immunoglobulins, antibiotics, interleukins (e.g. IL-2, IL-12), and/or cytokines (e.g. IFNγ).

In one embodiment, the immunogenic compositions, therapeutic formulations, medicaments, pharmaceutical compositions, and prophylactic formulations (e.g. vaccines) of the invention can be administered to a subject such as a mammal (e.g. a human, bovine, porcine, ovine, caprine, equine, cervine, canine or feline subject) in conjunction with (simultaneously or sequentially) one or more antibiotic compounds.

The immunogenic compositions, therapeutic formulations, medicaments, pharmaceutical compositions, and prophylactic formulations (e.g. vaccines) may contain 5% to 95% of active ingredient, such as at least 10% or 25% of active ingredient, or at least 40% of active ingredient or at least 50, 55, 60, 70 or 75% active ingredient.

The immunogenic compositions, therapeutic formulations, medicaments, pharmaceutical compositions, and prophylactic formulations (e.g. vaccines) are administered in a manner compatible with the dosage formulation, and in such amount as will be prophylactically and/or therapeutically effective.

Administration of immunogenic compositions, therapeutic formulations, medicaments, pharmaceutical compositions, and prophylactic formulations (e.g. vaccines) is generally by conventional routes e.g. intravenous, subcutaneous, intraperitoneal, or mucosal routes. The administration may be by parenteral administration; for example, a subcutaneous or intramuscular injection.

Accordingly, immunogenic compositions, therapeutic formulations, medicaments, pharmaceutical compositions, and prophylactic formulations (e.g. vaccines) of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the immunogenic compositions, therapeutic formulations, medicaments, pharmaceutical compositions, and prophylactic formulations (e.g. vaccines) may contain minor amounts of auxiliary substances such as wetting or emulsifying agents, and/or pH buffering agents.

Generally, the carrier is a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long term storage.

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

It may be desired to direct the compositions of the present invention (as described above) to the respiratory system of a subject. Efficient transmission of a therapeutic/prophylactic composition or medicament to the site of infection in the lungs may be achieved by oral or intra-nasal administration.

Formulations for intranasal administration may be in the form of nasal droplets or a nasal spray. An intranasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

Alternatively, the therapeutic/prophylactic formulation or medicament may be an aerosol formulation. The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

In one embodiment, the immunogenic compositions, therapeutic formulations, medicaments, pharmaceutical compositions, and prophylactic formulations (e.g. vaccines) of the invention comprise a pharmaceutically acceptable carrier, and optionally one or more of a salt, excipient, diluent and/or adjuvant.

In one embodiment, the immunogenic compositions, therapeutic formulations, medicaments, pharmaceutical compositions, and prophylactic formulations (e.g. vaccines) of the invention may comprise one or more immunoregulatory agents selected from, for example, immunoglobulins, antibiotics, interleukins (e.g. IL-2, IL-12), and/or cytokines (e.g. IFNγ).

The present invention encompasses polypeptides that are substantially homologous to polypeptides based on any one of the polypeptide antigens identified in this application (including fragments thereof). The terms “sequence identity” and “sequence homology” are considered synonymous in this specification.

By way of example, a polypeptide of interest may comprise an amino acid sequence having at least 70, 75, 80, 82, 84, 86, 88, 90, 92, 94, 96, 98, 99 or 100% amino acid sequence identity with the amino acid sequence of a reference polypeptide.

There are many established algorithms available to align two amino acid sequences.

Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g. GAP, BESTFIT, FASTA or TFASTA), or BLAST and BLAST 2.0 algorithms.

The BLOSUM62 table shown below is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915-10919, 1992; incorporated herein by reference). Amino acids are indicated by the standard one-letter codes. The percent identity is calculated as:

$$\frac{Total number of identical matches}{[ \begin{matrix}length of the longer sequence plus the number of gaps \\ Introduced into the longer \\ sequence in order to align the two sequences\end{matrix} ]} \times 100$$

BLOSUM62 TABLE

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

In a homology comparison, the identity may exist over a region of the sequences that is at least 10 amino acid residues in length (e.g. at least 15, 20, 30, 40, 50, 75, 100, 150, 200, 250, 300, 350, 400, 450, 500, 550, 600, 650 or 685 amino acid residues in length—e.g. up to the entire length of the reference sequence.

Substantially homologous polypeptides have one or more amino acid substitutions, deletions, or additions. In many embodiments, those changes are of a minor nature, for example, involving only conservative amino acid substitutions. Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous polypeptides also encompass those comprising other substitutions that do not significantly affect the folding or activity of the polypeptide; small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

The polypeptides of the invention may also comprise non-naturally occurring amino acid residues. In this regard, in addition to the 20 standard amino acids, non-standard amino acids (such as 4-hydroxyproline, 6-N-methyl lysine, 2-aminoisobutyric acid, isovaline and α-methyl serine) may be substituted for amino acid residues of the mycobacterial polypeptides of the present invention. A limited number of non-conservative amino acids, amino acids that are not encoded by the genetic code, and unnatural amino acids may be substituted for mycobacterial polypeptide amino acid residues. Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine.

Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations can be carried out in a cell free system comprising an E. coli S30 extract and commercially available enzymes and other reagents. Peptides can be, for instance, purified by chromatography. In a second method, translation is carried out in Xenopus oocytes by microinjection of mutated mRNA and chemically aminoacylated suppressor tRNAs. Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. Naturally occurring amino acid residues can be converted to non-naturally occurring species by in vitro chemical modification. Chemical modification can be combined with site-directed mutagenesis to further expand the range of substitutions.

Essential amino acids, such as those in the polypeptides of the present invention, can be identified according to procedures known in the art, such as site-directed mutagenesis or alanine-scanning mutagenesis. Sites of biological interaction can also be determined by physical analysis of structure, as determined by such techniques as nuclear magnetic resonance, crystallography, electron diffraction or photoaffinity labelling, in conjunction with mutation of putative contact site amino acids. The identities of essential amino acids can also be inferred from analysis of homologies with related family members of the polypeptide of interest.

Multiple amino acid substitutions can be made and tested using known methods of mutagenesis and screening. Methods are known for simultaneously randomizing two or more positions in a polypeptide, selecting for functional polypeptide, and then sequencing the mutagenized polypeptides to determine the spectrum of allowable substitutions at each position. Other methods that can be used include phage display.

Routine deletion analyses of nucleic acid molecules can be performed to obtain functional fragments of a nucleic acid molecule that encodes a polypeptide of the invention. As an illustration, DNA molecules can be digested with Bal31 nuclease to obtain a series of nested deletions. These DNA fragments are then inserted into expression vectors in proper reading frame, and the expressed polypeptides are isolated and tested for the desired activity. An alternative to exonuclease digestion is to use oligonucleotide-directed mutagenesis to introduce deletions, or stop codons to specify production of a desired fragment. Alternatively, particular polynucleotide fragments can be synthesized using the polymerase chain reaction.

A mutant of a polypeptide of the invention may contain one or more analogues of an amino acid (e.g. an unnatural amino acid), or a substituted linkage, as compared with the sequence of the reference polypeptide. In a further embodiment, a polypeptide of interest may be a mimic of the reference polypeptide, which mimic reproduces at least one epitope of the reference polypeptide.

Mutants of the disclosed polynucleotide and polypeptide sequences of the invention can be generated through DNA shuffling. Briefly, mutant DNAs are generated by in vitro homologous recombination by random fragmentation of a parent DNA followed by reassembly using PCR, resulting in randomly introduced point mutations. This technique can be modified by using a family of parent DNAs, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed above can be combined with high-throughput screening methods to detect activity of cloned mutant polypeptides. Mutagenized nucleic acid molecules that encode polypeptides of the invention, or fragments thereof, can be recovered from the host cells and rapidly sequenced using modern equipment. These methods allow the rapid determination of the importance of individual amino acid residues in a polypeptide of interest, and can be applied to polypeptides of unknown structure.

A “fragment” of a polypeptide of interest comprises a series of consecutive amino acid residues from the sequence of said polypeptide. By way of example, a “fragment” of a polypeptide of interest may comprise (or consist of) at least 10 consecutive amino acid residues from the sequence of said polypeptide (e.g. at least 15, 20, 25, 28, 30, 35, 40, 45, 50, 55, 75, 100, 125, 150, 175, 200, 225, 250, 275, 300, 325, 350, 375, 400 or 412 consecutive amino acid residues of said polypeptide). A fragment may include at least one epitope of the polypeptide of interest.

A polypeptide of interest, or fragment, may possess the active site of the reference polypeptide.

The polypeptide of interest, or fragment thereof, may have a common antigenic cross-reactivity and/or substantially the same in vivo biological activity as the reference peptide. For example, the polypeptides, or polypeptide fragments, and reference polypeptides share a common ability to induce a “recall response” of a T-lymphocyte (e.g. CD4+, CD8+, effector T cell or memory T cell such as a TEM or TCM), which has been previously exposed to an antigenic component of a mycobacterial infection.

New immunological assays for measuring and quantifying T cell responses have been established over the last 10 years. For example, the interferon-gamma (IFN-γ) ELISPOT assay is useful as an immunological readout because the secretion of IFN-γ from antigen-specific T cells is a good correlate of protection against M. tuberculosis. Furthermore, the ELISPOT assay is a very reproducible and sensitive method of quantifying the number of IFN-γ secreting antigen-specific T cells.

As used herein, the terms “nucleic acid sequence” and “polynucleotide” are used interchangeably and do not imply any length restriction. As used herein, the terms “nucleic acid” and “nucleotide” are used interchangeably. The terms “nucleic acid sequence” and “polynucleotide” embrace DNA (including cDNA) and RNA sequences.

The polynucleotide sequences of the present invention include nucleic acid sequences that have been removed from their naturally occurring environment, recombinant or cloned DNA isolates, and chemically synthesized analogues or analogues biologically synthesized by heterologous systems.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 82, 84, 86, 88, 90, 92, 94, 96, 98 or 99% of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different Thr codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

Thus, in one embodiment of the invention, the nucleic acid sequence is codon optimized for expression in a host cell.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest.

### FIGURE LEGENDS

FIG. 1a-c. Protective impact of MVA single dose regimes expressing BitC.

Balb/C mice were immunised with Modified Vaccinia Ankara 10⁶ pfu on day 0. The vectors either expressed antigens, or had empty antigen expression cassettes. Animals receiving IsdA encoding vector also received 20 μg of ClfB protein in adjuvant simultaneously. Two weeks later, the animals were challenged with S. aureus strain Newman intravenously. S. aureus was enumerated in the kidneys 3 days later. Luciferase immunoprecipitation was used to monitor antibody production.

FIG. 2a-c. Protective impact of adenovirus prime, MVA boost regimes expressing BitC (i).

Balb/C mice were immunised with 10⁹ i.u. adenovirus Hu5 on day 0, and Modified Vaccinia Ankara 10⁷ pfu on day 56. The vectors either expressed antigens, or had empty antigen expression cassettes. Animals receiving IsdA encoding MVA also received 20 μg of ClfB protein in adjuvant simultaneously. Two weeks later, the animals were challenged with S. aureus strain Newman intravenously. S. aureus was enumerated in the kidneys 3 days later. Luciferase immunoprecipitation was used to monitor antibody production. ELISpot assay was used to enumerate IFN-gamma producing cells.

FIG. 3a-e. Protective impact of adenovirus prime, MVA boost regimes expressing BitC (ii).

Balb/C mice were immunised with 10⁹ i.u. adenovirus Hu5 on day 0, and Modified Vaccinia Ankara 10⁷ i.u. on day 56 as illustrated in FIG. 3a. The vectors either expressed antigens, or had empty antigen expression cassettes.

FIG. 3b shows the background subtracted luciferase activity following immunoprecipitation of antigen-renilla luciferase fusions by antisera generated in the immunised animals (log₁₀ LU-BG), which is a measure of antibody induction. The sera used were taken immediately pre-challenge. The left panel shows pooled results from five independent experiments. ‘Vector’ refers to pulldown of BitC-renilla luciferase antigen fusion by sera from animals immunised by viral vectors without antigen. ‘BitC’ refer to pulldown of BitC-renilla luciferase antigen fusion by sera from animals immunised by viral vectors expressing BitC.

The right panel shows log₁₀ LU-BG following pulldown of BitC-renilla fusion protein from control (viral vectors without antigen, black dots) and BitC (viral vectors with BitC antigen, red dots) groups for each of five individual experiments; experiment codes are on the x-axis.

FIG. 3c shows the number of interferon-gamma secreting cells in blood taken immediately pre-challenge, as assessed by ELISPOT. The left panel shows pooled results from four independent experiments. ‘Vector’ refers to spot numbers from blood from animals immunised by viral vectors without antigen, while ‘BitC’ refers to spot numbers from blood from animals immunised by viral vectors expressing BitC. In both cases, stimulation was with a pool of overlapping peptides spanning the BitC protein.

The right panel shows results from four independent experiments in which blood interferon-gamma ELISPOT was performed. Interferon-gamma secreting cell numbers are shown following stimulation with a peptide pool of peptides overlapping the BitC protein following immunisation with viral vectors without antigen, (black dots) or viral vectors with BitC antigen (red dots). Experiment codes are on the x-axis.

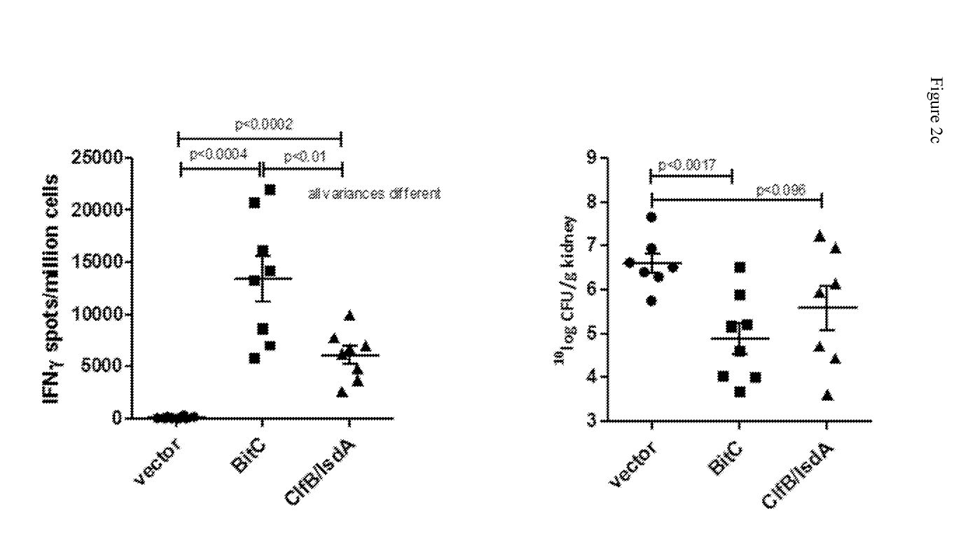

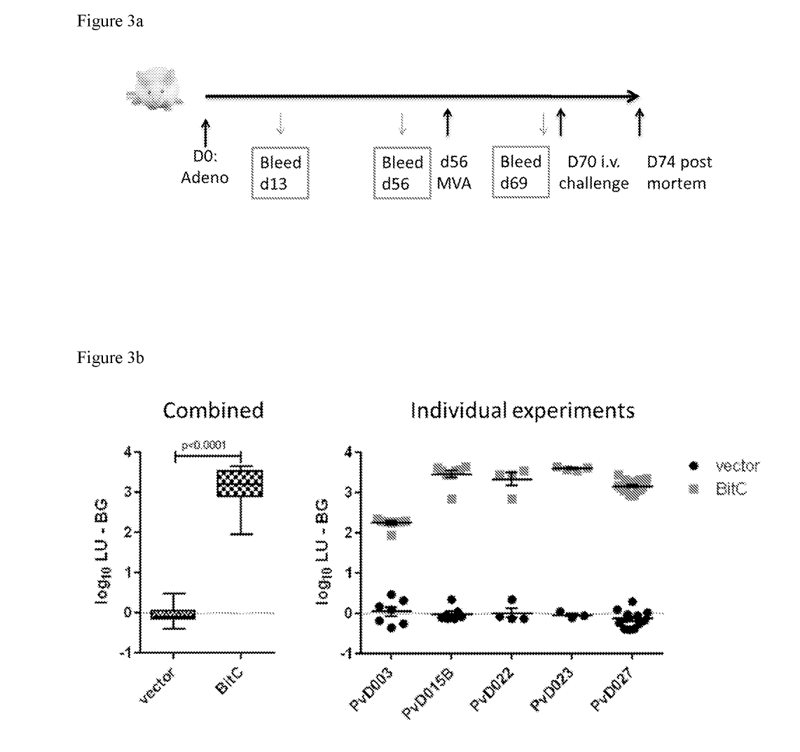

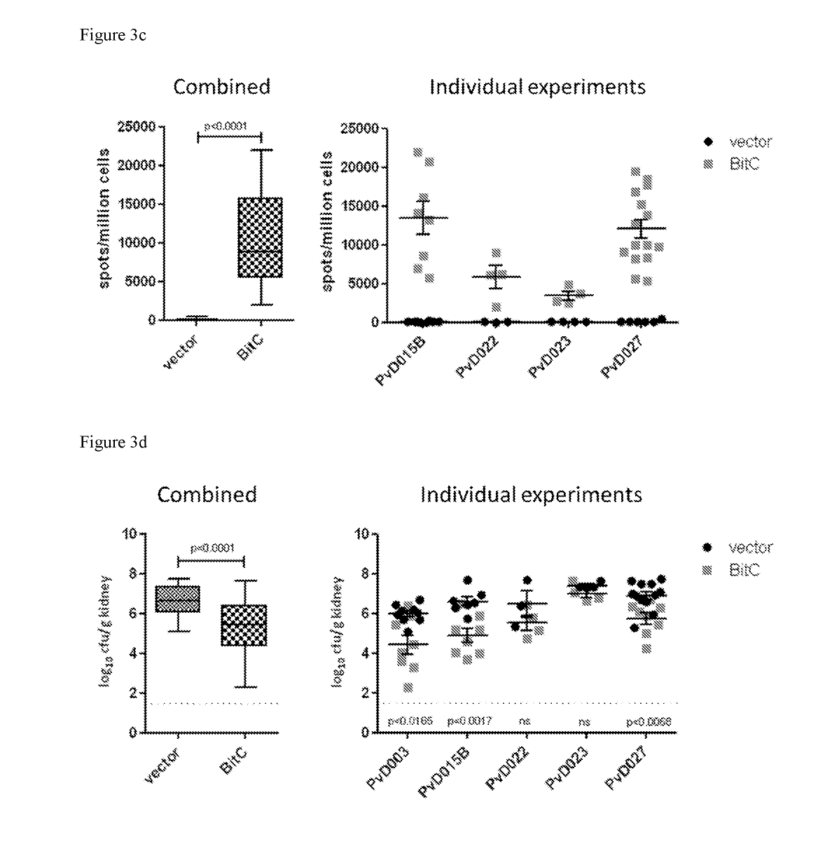

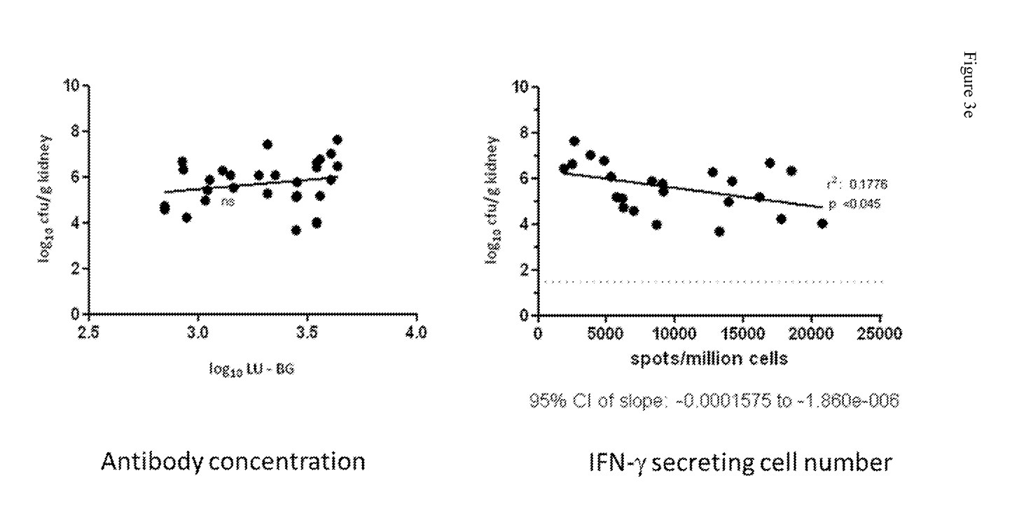

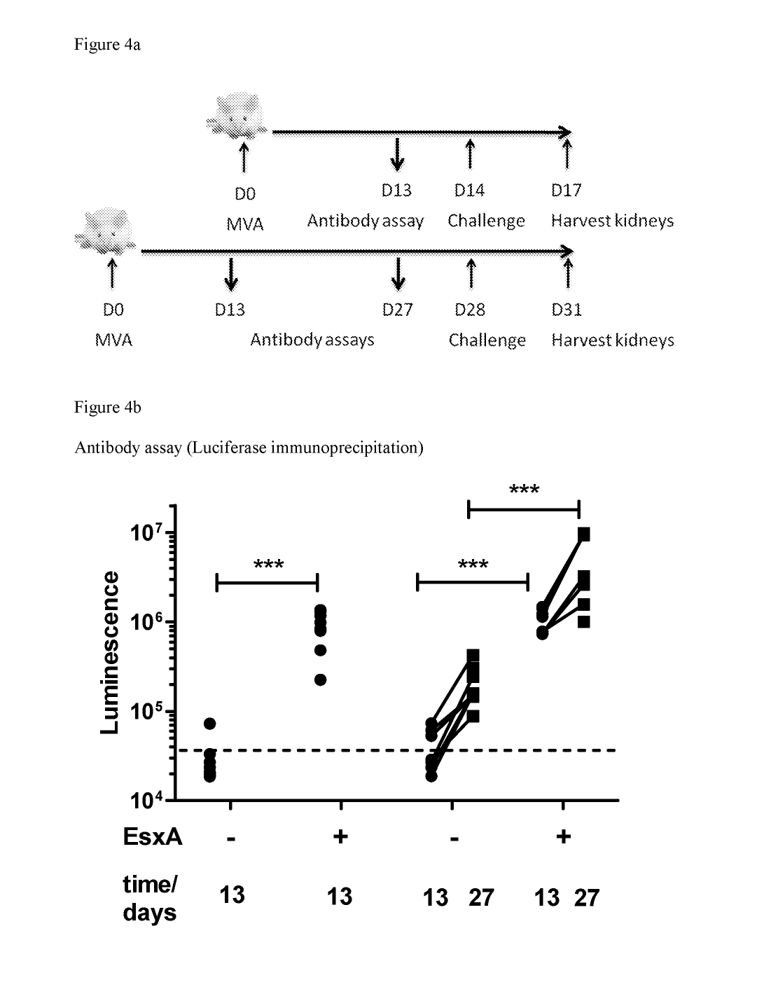

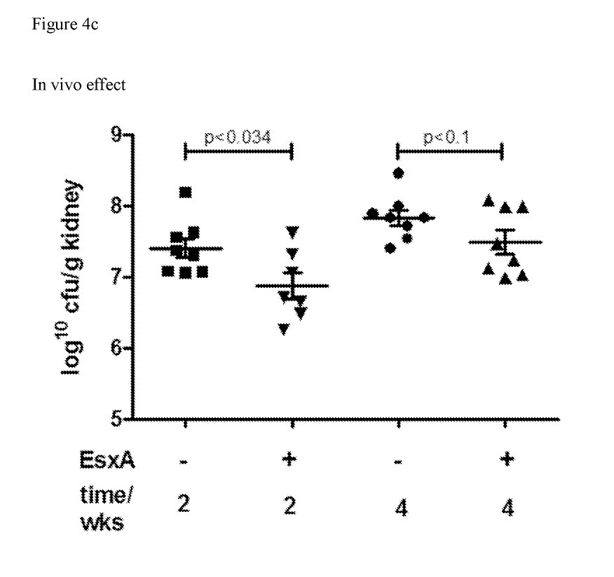

Subsequently, the animals were challenged with S. aureus strain Newman intravenously. S. aureus was enumerated in the kidneys 3 days later (FIG. 3d) Bacterial counts recovered following vaccination with control vectors (no antigen), or BitC are shown. On the right, results from five separate experiments are shown. In FIG. 3e, the association between pre-challenge antibody response (left hand panel) and pre-challenge interferon-gamma T cell numbers is shown on a mouse by mouse basis.

FIG. 4a-c. Protective impact of single dose MVA expressing EsxA.

Balb/C mice were immunised with a single dose of 10⁶ pfu Modified Vaccinia Ankara on day 0. The vectors either expressed EsxA, or had empty antigen expression cassettes. Animals receiving IsdA encoding vector also received 20 μg of ClfB protein in adjuvant simultaneously. Either two or four weeks later, the animals were challenged with S. aureus strain Newman intravenously. S. aureus was enumerated in the kidneys 3 days later. Luciferase immunoprecipitation was used to monitor antibody production.

### KEY TO SEQ ID NOS

SEQ ID NO: 1 DNA sequence of S. aureus polypeptide BitC
SEQ ID NO: 2 DNA sequence of S. aureus polypeptide NWMN_{—}2593
SEQ ID NO: 3 DNA sequence of S. aureus polypeptide NMWN_{—}2585
SEQ ID NO: 4 DNA sequence of S. aureus transposition regulatory polypeptide tnpC
SEQ ID NO: 5 DNA sequence of S. aureus urease accessory polypeptide UreD
SEQ ID NO: 6 DNA sequence of S. aureus polypeptide NMWN_{—}2109
SEQ ID NO: 7 DNA sequence of S. aureus galactose-6-phosphate isomerase subunit LacB
SEQ ID NO: 8 DNA sequence of S. aureus SA1633
SEQ ID NO: 9 DNA sequence of S. aureus NWMN_{—}1623
SEQ ID NO: 10 DNA sequence of S. aureus tnp
SEQ ID NO: 11 DNA sequence of S. aureus polypeptide NWMN_{—}0254
SEQ ID NO: 12 DNA sequence of S. aureus polypeptide NWMN_{—}0257
SEQ ID NO: 13 DNA sequence of S. aureus polypeptide SbnB
SEQ ID NO: 14 DNA sequence of S. aureus MW0751
SEQ ID NO: 15 DNA sequence of S. aureus polypeptide SA0193
SEQ ID NO: 16 DNA sequence of S. aureus polypeptide NMWN_{—}1106
SEQ ID NO: 17 DNA sequence of S. aureus polypeptide EsxA (NMWN_{—}0219)
SEQ ID NO: 18 Amino acid sequence of S. aureus polypeptide BitC
SEQ ID NO: 19 Amino acid sequence of S. aureus polypeptide NWMN_{—}2593
SEQ ID NO: 20 Amino acid sequence of S. aureus polypeptide NMWN_{—}2585
SEQ ID NO: 21 Amino acid sequence of S. aureus transposition regulatory polypeptide tnpC
SEQ ID NO: 22 Amino acid sequence of S. aureus urease accessory polypeptide UreD
SEQ ID NO: 23 Amino acid sequence of S. aureus polypeptide NMWN_{—}2109
SEQ ID NO: 24 Amino acid sequence of S. aureus polypeptide galactose-6-phosphate isomerase subunit LacB
SEQ ID NO: 25 Amino acid sequence of S. aureus SA1633
SEQ ID NO: 26 Amino acid sequence of S. aureus NWMN_{—}1623
SEQ ID NO: 27 Amino acid sequence of S. aureus tnp
SEQ ID NO: 28 Amino acid sequence of S. aureus polypeptide NWNM_{—}0254
SEQ ID NO: 29 Amino acid sequence of S. aureus polypeptide NMWN_{—}0257
SEQ ID NO: 30 Amino acid sequence of S. aureus polypeptide SbnB
SEQ ID NO: 31 Amino acid sequence of S. aureus MW0751
SEQ ID NO: 32 Amino acid sequence of S. aureus polypeptide SA0193
SEQ ID NO: 33 Amino acid sequence of S. aureus polypeptide NMWN_{—}1106
SEQ ID NO: 34 Amino acid sequence of S. aureus polypeptide (EsxA) NMWN_{—}0219
SEQ ID NO: 35 DNA sequence of EsxA-V5-313 fusion
SEQ ID NO: 36 DNA sequence of BitC.V5.313 fusion
SEQ ID NO: 37 DNA sequence of EsxA.rLuc
SEQ ID NO: 38 DNA sequence of BitC.rLuc
SEQ ID NO: 39 DNA sequence of pMono2.BitC.313
SEQ ID NO: 40 DNA sequence of pMVA.BitC.313

| Sequences | |
|---|---|
| SEQ ID NO: 1 | |
| - BitC | |
| Atgaaatcaaaaatttatatcttgctattatttctcatttttttatcagcatgcgctaatacgcgtcactctgaatccgataaaaatg | |
| tattaacagtttattctccgtatcaatcaaacttgattcgtccaattttaaatgaatttgaaaaacaagagcatgtcaaaattgaaa | |
| ttaaacacggatctactcaagtactgctttcaaacttgcataacgaagatttttcggagcgtggtgatgtctttatgggtggtgtg | |
| ttgtcagaaacaattgatcatccagaagattttgttccctatcaagatacatctgtaacacagcaattagaggattatcgctcga | |
| acaataaatatgttactagttttctattaatgccaacagttatagtagtgaattcagatttacaaggagatattaagattcgaggtt | |
| atcaagatttattacaacctatacttaaaggtaaaattgcgtactcaaatccaaatacaacaacgacaggctatcaacatatgc | |
| gtgctatttatagcatgcatcatcgagtaagtgatgtgcatcaattccaaaaccatgcgatgcaactgtcaaagacgtctaaag | |
| tcattgaagatgttgcaaaaggtaaatattacgcaggtctaagctacgaacaagatgcacgcacatggaaaaacaaaggtta | |
| tcctgtatcaatcgtttatccaattgaaggaacaatgttaaatgttgatggtattgctttagttaaaaatgcacaccctcatcctaa | |
| gcgtaaaaagttagtgcaatatttaacaagccgctcagtacaacaacgattagttgcagagttcgatgccaagtcaattcgaa | |
| aggatgttagtgaacaaagcgatcagtctatcgaaaatcttaagaacatacctttaataccgaaatctaaattaccggatattc | |
| cacatcataaatttttggagatgattcaatga | |
| SEQ ID NO: 2 | |
| - NWMN 2593 | |
| Atgaattcagagtataaaaaaggcatatttttagcactcagtgcatacattctgtggggaatactacctatatattggcagttcg | |
| ttgatgcaataggcgcatttgaaattttagcctttcgtattatattttcagcaatattcatgattttcatactcgcggttggacaaaa | |
| acaacgcaatgcatttcaacgagatatgaatcaattgttaggcaagcccattcagctattagcgattgtcgtagcaggctatgt | |
| cattacattaaattggggtacatttatttgggctgtaacgaacggtcacgtcctacaaacaagtttaggttattatataaatccac | |
| ttgttagcattttgctcgcacttatctttttaaaagaaagattcaataaatttgaatggctagccattttattcgcattcatcggtgtat | |
| tatatatgacgctcaagattggagaattcccaatcgtctctattatattagcgttatcctttggtacatacggattattgaaaaaag | |
| tagtacatattgatgccatcagcagtattacgattgaatgtattgttaccgcacctgctggactaatatacgttatttatttatggc | |
| agcaacatcagatgtcatttggattgaacatgtcatcattttggttgttattttctggtgctattacggcaataccactaatcctatt | |
| ctcagccggggcaaaacgtattccactttcgctaataggatttattcaatacgttggaccaacaataatgtttgtactcggcata | |
| tttgttttcaaagagccttttagtatagatcaattaattacgtttatatttatttggacaggtattgtgttatatagtctttctcaatacat | |
| taaattgaagaaacatccggtcgcaaaaaccctataa | |
| SEQ ID NO: 3 | |
| NMWN_2585 | |
| Atgactaactttacttttgatggtgcacacagtagtttagaattccaaattaaacatttaatggtttctaaagtgaaaggttcattt | |
| gatcaatttgatgtagctgttgaaggagatattaatgacttcagtactttgaaagctactgcaacaattattccaagctcaattaa | |
| cactaaaaacgaagcacgtgataaccacttaaaatctggtgatttctttggtactgacgaatttgataaaattacatttgtgaca | |
| aaatcagtatctgaaagcaaagttgttggtgatttaacaattaaaggcatcactaacgaagaaacattcgatgttgaattcaac | |
| ggagtaagtaagaatcctatggatggttctcaagtaacaggtattattgttactggtacaatcaatagagaaaaatatggcatt | |
| aactttaaccaagcacttgaaactggtggcgtaatgctaggcaaagatgttaaattcgaagcatcagctgaattctcaatctca | |
| gaataa | |
| SEQ ID NO: 4 | |
| tnpC | |
| atggataaacaagttagaaatacaacagaaattgtacgtttggcgaagcagaaatcaaaaaagacaagggaaaaagtaga | |
| caaagcgatttctaaattttcgattgaaggtaaagttattaattttaattcaatagcaaaggaagctaatgtttctaaatcatggctt | |
| tataaggaacacgatattaggcaaagaatcgaatcccttcgtgagcgtcaaataacagcaaatgtagtctcaaaacccaaga | |
| aaagttctcgttcggaggaaatccttattaaaaccttaaaaagaagagtaatggaattagaaaaagaaaataaaaaattacag | |
| aaccaaattcaaaaattatatggagatctgtataataaagaataa | |
| SEQ ID NO: 5 | |
| UreD | |
| Atggatgaacaacaatggactgggcaacttgatttaacagtgtttttcgatggcaatcgatcagtatcaagagatattttctttg | |
| aaaaagcacttaaagtgatacgtccagtttatctaaatcaatctaccattcctacattttatatagtaaatgtaggtggtggctatt | |
| tagatggagatcgttaccgtatgaatgtgaatgtcgaagataacgctaaagtgacattgacatctcaaggtgcaacaaaaata | |
| tacaagacaccttccaatcatgttgagcagtatcaaacttttaatttgaaagataacgcatatttagaatatgtcgctgatccaat | |
| catcgcatatgaaaatgctaaattttatcaacacaatacgttcaatctcaataattctagttcattattttatactgatattttaactcc | |
| tggttattcaaaaactggcgaagccttcaagtatcaatatatgcatttaataaatgaaatttatattgaagatgagttagtcacata | |
| tgataatttattattgaatcctaataaacaatcgatcaatgaaataggttatatggaacattactctcattatggctctgcttacttc | |
| atacacgaagatgttaaccaaaaactaatcgattcagtttatgaaaccattagttcttatagcaatacattcgattgtcgtgttgct | |
| atttctcaattgcctacacatggttttgcagttagaatttttgcttatcgcacacaaattatagagaaaatacttggaaccatccaa | |
| agctatatcgcagaaaatatttatgatcgtaaacttgattttctaagaaaatattaa | |
| SEQ ID NO: 6 | |
| NMWN_2109 | |
| Atgaaactaaaatcatttgttactgccactttagcattgggattattatcaacggtcggagctgcattaccgagtcacgaagca | |
| tctgcagatagtaataacggctataaagaaatgactgtggatggttatcacactgttccttacacaatttcagtagatggtatta | |
| ctgcattacatcgaacttactttatcttcccagaaaataaaaatgttctttatcaagaaattgacagtaaagtaaaaaatgaatta | |
| gcttctcaacgtggtgttacaacagaaaaaattaataatgcccaaacagcaacttatacgcttactttgaatgatggtaataaa | |
| aaagtagtgaatctaaagaaaaatgacgacgctaaaaattcaattgatccaagtacaatcaaacagatacaaattgtagttaa | |
| ataa | |
| SEQ ID NO: 7 | |
| LacB | |
| Atgaagattgcattaggatgcgaccatattgttacagatacaaaaatgcgtgtatctgaatttttaaaatcaaaaggacatgaa | |
| gtcattgacgtaggaacatacgatttcacaagaacacattatccaatttttggtaaaaaagttggcgaacaagttgttagcggt | |
| aatgcagacttaggtgtttgtatttgtggaacaggtgttggtattaacaatgctgtaaataaagtacctggcgttcgttcagcact | |
| agtacgtgatatgacatcagcgttatacgctaaagaagaattaaatgcgaacgttattggcttcggtggacgtattataggtga | |
| gttattaatgtgcgatattatcgatgcatttattaatgctgaatataaaccaactgaagagaacaaaaaattaatcgctaaaatta | |
| aacatttagaaacaagcaatgcagatcaagctgatccacatttctttgatgaattcttagaaaaatgggacagaggcgaatac | |
| cacgattaa | |
| SEQ ID NO: 8 | |
| SA1633 | |
| Atgaatacaaaatttttaggtaaaacattagtagcaagtgctttagtattaacaacattaggaacaggtttacattcttcatactta | |
| ggattaaatacaaataaagttgttaaaacagcaaaagcagaagaaaaaatgacaaatggtcaattgtggaaaaaagttaaa | |
| gattcattaattgattcaaatattattagtggtaacgaaaatgaagagattacagtaacatatgttaataaaaccggatattcttca | |
| agtgtttctgcctatggtaataataatgacgatttttcaagtactccaagtaatttttctaaacttaaggaaattgacttgaaaaaa | |
| gataatgtaccgtcagatgattttaatactactgttagtggtgaagatagttggaaaacacttacatctaaattaaaggaaaaag | |
| gtttggttacagatggacaaacagtaactattcattgtaatgataagtctgacaatacaaaatcatctgtttcaggtaaagtagg | |
| agcagatctgacaagtggcaatggaactacattcaaaaaacgctttattgataaaatcacaattgattaa | |
| SEQ ID NO: 9 | |
| NWMN_1623 | |
| Atgacgaatcaagacaacaatcatcaattgaatcatcgtatatatcattttgaaaagatatataaagctatcaaacatgtcattg | |
| tttttatatttatgattttcattgccatcgttgctatcgctgtgattgcgatgtctttatattttcatcatttaactaaaacgtccgactca | |
| ttatcagatgatgctttaataaaaaaagttcgacaaatacctggcgatgaattattagatcataataacaaaaatttattatatga | |
| gtataaccattctcaaaactcactcattataggccctaaaacatcaagtccaaatgtcattaaagcattaacgtcatctgaagac | |
| actttattttataaacatgatggcatcttaccaaaggcgattttaagagcaatgatacaagatatttttaatactgatcaaagttca | |
| ggtggtagcacaattacacaacaacttgttaaaaatcaagttcttaccaacgaaaaaacatatagtagaaaagcaaatgaact | |
| tcgcctagcaattagattagaacacctactctcaaaagatgaaattatatatacatatttaaatatagttcccttcggtagagatta | |
| taatggcgctaatatttccggaattgcatccgcttcatatagtctatttggtattccaccaaaagatttatcaattgcacaatctgc | |
| ataccttatcggtttgttgcaaagcccatatggctatacaccctacgaaaaagatggaacgttaaaatcggataaagatttgaa | |
| atatagtattcaaagacaacattatgtattaaagcgtatgttaatcgaagatcaaatcactgaaaaagaatacaacgacgcatt | |
| aaaatatgatattaaatcacatttgttaaatcgaaaaaagcgttaa | |
| SEQ ID NO: 10 | |
| tnp | |
| Attttgagtttcactcgaatgtcagttcgaggaataaataaagttaaacgagagctaggttttgtattaatggcacttaatataa | |
| ggaaaatagcagctcaacgagctgtacattataaaatacatatcaaaaaagctgatttctatcaaataattaatagaaatcagc | |
| tttttacattgcctaagaacttaatgtcccagcctccttcataa | |
| SEQ ID NO: 11 | |
| NWMN_0254 | |
| atggcaaatttacaaaagtatattgagtattctcgagaagttcagcaagcacgggagaacaatcaaccgattgtagcattaga | |
| atcaacaattatttcgcatggtatgccgtacccacaaaatgttgaaatggcaacaacagtagagcaaattatcaggaataatg | |
| gtgccattccagcaaccatagccattatagatggcaaaattaaaattggtttagaaagcgaagatttagaaatactggcaact | |
| agtaaagacgttgctaaagtatctagaagggatttagcagaagttattgcgatgaagtgtgttggtgctactactgtagcgac | |
| gacgatgatatgtgctgcaatggctggtattcaattttttgttacaggaggtattgggggcgtccataaaggtgcagaacatac | |
| gatggacatttcagcagacttagaagaactgtctaaaacaaatgtcactgttatctgtgcaggtgccaaatcaattttagactta | |
| cctaagacgatggagtatttagaaacaaaaggcgttccagttattggatatcaaacgaatgaattgccagcattcttcactcgc | |
| gaaagcggtgttaagttaacaagttcggttgaaacgccagaacgacttgctgacattcatttaacaaaacagcagttaaatctt | |
| gaaggtggcattgttgttgctaatccaattccatatgagcatgccttatcaaaagcatatattgaggcaatcataaatgaagctg | |
| ttgttgaageggaaaatcaaggtattaaaggtaaggacgccacaccgttcttgttagggaaaattgtagaaaaaacgaatgg | |
| taaaagtttagcagcaaatataaaacttgttgaaaacaatgcggcgttgggtgctaaaattgctgtcgctgttaataaattattgt | |
| ag | |
| SEQ ID NO: 12 | |
| NWMN_0257 | |
| Ttgacaagctttatctataaaatactttatgttgtcaaaatcaacgcttatacatatgacataatgacggaggacatcatgattct | |
| atctatattacttatctttttctgtatcagacttgtcagcttaaagatatctattaatcactcaaaacaattaaaagcagacggtgca | |
| gttgaatatggcgttaaaaattcgaagtttttagcaataacacacgttttaatttatgtattggctggtgtagaggcatttattaata | |
| aagatacatttagctttgcaaatggtattggcttagttatattaatctttgcttatatcatgttatttatggttattaaaactttaggtgg | |
| tatttggacattaaaattattcattttacctaaccatcctattattaaatctggattatataaaattacaaaacacccaaattacttctt | |
| aaacatcattcctgaattaatcggtgtattattattaacacacgcaacatatacaactattttattagtaccgtatgcgtatttcttat | |
| atgtacgtattaaacaagaagagaaattaatgaatatttaa | |
| SEQ ID NO: 13 | |
| SbnB | |
| atgaatagagagatgttgtatttaaatagatcagatattgaacaagcgggaggtaatcattcacaagtttatgtggacgcatta | |
| acagaagcattaacagcccatgcgcacaatgattttgtacaaccgcttaagccgtatttaagacaggatcctgaaaatggac | |
| acatcgcagatcgaattattgcaatgccaagtcatatcggtggtgaacacgcaatttcaggtattaagtggataggtagtaag | |
| cacgacaatccatcgaaacgtaatatggagcgtgcaagtggcgtcattattttgaatgatccagaaacgaattatccaattgc | |
| agttatggaagcaagtttaattagtagtatgcgtactgcagcagtttcagtgattgcagcaaagcatttggctaaaaaaggattt | |
| aaagacttaacaatcattggatgegggctaatcggagacaagcaattacaaagtatgttagagcaattcgatcatattgaacg | |
| cgtgtttgtttacgatcaattctctgaagcatgtgcacgctttgttgatagatggcaacaacagcgtccggaaattaattttattg | |
| cgacagaaaatgctaaagaagcagtatcaaatggtgaagtagtcattacatgtaccgtaacggatcaaccatacattgaatat | |
| gattggttacaaaagggtgcatttattagcaacatttctatcatggatgtgcataaagaagtctttattaaagctgacaaagtcgt | |
| agtagatgactggtcacaatgtaatcgagaaaagaaaactattaaccaattggtgttagaaggtaaattcagcaaagaagct | |
| cttcatgctgaactaggacaacttgtgacaggtgacataccaggacgtgaagacgatgatgagatcatattacttaatccgat | |
| gggtatggctatcgaagatatttcaagtgcttattttatttatcaacaggcacaacaacaaaatattgggacaacattgaaccta | |
| tattaa | |
| SEQ ID NO: 14 | |
| MW0751 | |
| gtggcaattaagaaaaaagatagaattattggagttaaagaattagaaataccgcaagaattaaagttagtgcctaattgggt | |
| attatggcgagctgaatggaacgagaagcaacaaaattatggaaaagtaccatatagtattaatggttacagagctagtaca | |
| accaataaaaaaacatggtgtgactttgaaagtgtaagtattgaatatgaagttgatgagcaatatagcggtataggttttgtat | |
| taagtgatggtaataattttgtttgcctagatattgataatgcaattgatgaaaaaggacaaatcaattctgaattagcattaaaa | |
| atgatgcgactcacatattgtgaaaagtctccaagtggtacaggattacattgtttctttaaaggtaaactaccagataaccgta | |
| aaaagaaaagaacggatttagacatagagttatatgattcagcaaggtttatgactgttacaggatgcacaattggtcaaaat | |
| gatatttgtgataatcaggaagtattaaatactctcattgatgaatactttaaagagaatttgccagtaaatgatgttgtgagaga | |
| ggaatctaatactaatatgcaattatctgatgaagatattataaacattatgatgaaatctaaacaaaaagataaaattaaagatc | |
| ttttacaaggcacatatgaatcatattttgacagttcgagcgaagcagtacaaagcttattacattatttagcgttttacacaggt | |
| aaaaacaaacagcaaatggagcgtatatttttaaactacaataatcttacggataaatgggaaagtaaacgaggtaatacga | |
| cttggggacagcttgagttagataaagctataaagaatcaaaagacagtttacactaaatccatagatgaatttaatgttataca | |
| acaggggagtaaagatgttaaacagttattgaatcaattggggcatgaagaaagaacaaaaatggaagaaaagtggattga | |
| aggaggaaaacgagggcgaaagcctacaacaattagccctataaaatgtgcatatattttgaatgagcatttaacatttatact | |
| ttttgatgatgaagaaaatactaagttagctatgtatcaatttgatgaagggatatatacacagaacactacaattataaaacga | |
| gtaatttcctatttagagcccaaacataatagtaataaagctgatgaagttatttatcatttaaccaatatggtagatataaaaga | |
| gaaaactaactcaccatacttaataccagttaaaaatggtgtatttaaccgtaaaacgaagcaactagaatcatttacacctga | |
| ttgtatatttaccactaaaatagatacatcgtatgtaaggcaagatatagtacctgaaataaatggctggaatatagatcggtgg | |
| atagaagaaatagcttgtaatgataatcaggttgttaaattattgtggcaagtaattaatgactcaatgaatggaaactacacac | |
| gtaaaaaagcaatatttttggttggtaatggtaacaatggtaaaggtacatttcaagaattattgtctaatgtaataggttatagc | |
| aatattgctagcttaaaagtgaatgagtttgatgaacgttttaaattgagtgtgttagagggcaagacagcagtaattggcgac | |
| gatgtaccagttggtgtgtatgtcgatgattcttcaaactttaaaagtgtagttactggcgatccagtgttggttgagtttaaaaat | |
| aaacccttatatagagcgacttttaagtgtacagttattcaatcaacgaatggaatgcctaaatttaaagacaaaacaggcgg | |
| gactttaagaaggttattgatagtaccgtttaatgccaattttaatggcattaaagagaattttaaaatcaaagaagactatataa | |
| aaaatccgcaagtgctagagtatgtgctttataaagcaattaatttagattttgaaacttttgacattcctgatgcatctgaaaaaa | |
| tgcttgaagtatttaaagaagataacgatccagtttatgggtttaaagtaaatatgtttgatcaatggactattagaaaagtgccg | |
| aaatatattgtatacgcattttataaagaatattgtgatgaaaatggctataatgcattgagttcaaacaagttttataaacaatttg | |
| aacattatttagagaattattggaaaactgatgcacagcgaagatatgacaatgaagaacttgctaagaggatatacaacttta | |
| atgacaatagaaattacattgaacctattgaaagtggaaaaaactataaatcgtatgaaaaggtgaagctaaaagcaatatag | |
| SEQ ID NO: 15 | |
| SA0193 | |
| Atgaggaaagttttaaatatttttacaatagcttttaaatcaatattgaaaaataaaggtagaaatatatttacaatgataggtata | |
| atcatcggtatttcttctgttattacgataatgtctttgggaaatggattaaaaaaactgctgctgatcaattttcagatgctggcg | |
| ccggaaaacaagaagcgttgattagctttacttttaaagttgatgagaaaatcaaaaagtatccttttaatcaaagagatataga | |
| gttggtaaatcaagttgatggcgtactagatgcgaagctaaaggaaaataaagaagaaggaattgaagccacaataactaa | |
| tgtccagaaaaaaagtgacatcttcattataaaaaaacaaaatttacactcttttaaagtaggtagagggtttgataaagaggat | |
| aatgaattacgtaagaaaatagtggttataaatgatcaagtagcaaaaactgtattcaataataatgctattggtaaatcgctat | |
| atattgagggacagggatttgaagtcataggaattacggataaactatattctgactcatctaccgttataatgcctgaaaatac | |
| attcaactactatatgggacacttacatcaaggtctgcctaccttacagataattattgaagatggttacaataaaaaaactgta | |
| gttaaaaaagtagaatcactattaaataaaaaaggatcgggctctgttttaggtgagtatacatatacagacactgaagaaatt | |
| atcaaaagtattgataaaatttttgatagtattacttattttgtagcagctgttgccggtatttcactttttattgctggtattggagtta | |
| tgaatgtcatgtatatatctgtagctgaaagaacagaagaaattgcgatacgacgtgcatttggtgcaaaaagtcgagatatt | |
| gaactacaatttttaatagagagcattttaatatgtgtgacaagtggttttattggacttattttaggtgttgtgtttgcaacaataat | |
| tgatgtattaacaccagattatattaaaagtgtagtaagtttgagttctgtcataattgcggttagtgtgtcgatattaattggactt | |
| ctttttggatggataccagctcgcgcagcatctaagaaagagctcatagatattataaaatag | |
| SEQ ID NO: 16 | |
| NMWN_1106 | |
| Ttgagtttcactcgaatgtcagttcgaggaataaataaagttaaacgagagctaggttttgtattaatggcaattaatataagg | |
| aaaatagcagctcaacgagctgtacattataaaatacatatcaaaaaagctgatttctatcaaataattaatagaaatcagctttt | |
| tacattgcctaagaacttaatgtcccagcctccttcataa | |
| SEQ ID NO: 17 | |
| EsxA (NMWN_0219) | |
| Atggcaatgattaagatgagtccagaggaaatcagagcaaaatcgcaatcttacgggcaaggttcagaccaaatccgtca | |
| aattttatctgatttaacacgtgcacaaggtgaaattgcagcgaactgggaaggtcaagctttcagccgtttcgaagagcaatt | |
| ccaacaacttagtcctaaagtagaaaaatttgcacaattattagaagaaattaaacaacaattgaatagcactgctgatgccgt | |
| tcaagaacaagaccaacaactttctaataatttcggtttgcaataa | |
| SEQ ID NO: 18 | |
| - BitC | |
| MKSKIYILLLFLIFLSACANTRHSESDKNVLTVYSPYQSNLIRPILNEFEKQEHV | |
| KIEIKHGSTQVLLSNLHNEDFSERGDVFMGGVLSETIDHPEDFVPYQDTSVTQ | |
| QLEDYRSNNKYVTSFLLMPTVIVVNSDLQGDIKIRGYQDLLQPILKGKIAYSN | |
| PNTTTTGYQHMRAIYSMHHRVSDVHQFQNHAMQLSKTSKVIEDVAKGKYY | |
| AGLSYEQDARTWKNKGYPVSIVYPIEGTMLNVDGIALVKNAHPHPKRKKLV | |
| QYLTSRSVQQRLVAEFDAKSIRKDVSEQSDQSIENLKNIPLIPKSKLPDIPHHKF | |
| LEMIQ | |
| SEQ ID NO: 19 | |
| - NWMN_2593 | |
| MNIEYKKGIFLALSAYILWGILPIYWQFVDAIGAFEILAFRIIFSAIFMIFILAVG | |
| QKQRNAFQRDMNQLLGKPIQLLAIVVAGYVITLNWGTFIWAVTNGHVLQTSL | |
| GYYINPLVSILLALIFLKERFNKFEWLAILFAFIGVLYMTLKIGEFPIVSIILALSF | |
| GTYGLLKKVVHIDAISSITIECIVTAPAGLIYVIYLWQQHQMSFGLNMSSFWLL | |
| FSGAITAIPLILFSAGAKRIPLSLIGFIQYVGPTIMFVLGIFVFKEPFSIDQLITFIFI | |
| WTGIVLYSLSQYIKLKKHPVAKTL | |
| SEQ ID NO: 20 | |
| - NMWN_2585 | |
| MTNFTFDGAHSSLEFQIKHLMVSKVKGSFDQFDVAVEGDINDFSTLKATATII | |
| PSSINTKNEARDNHLKSGDFFGTDEFDKITFETKSVTENKVVGDLTIKGITNEE | |
| TFDVEFNGVSKNPMDGSQVTGVIVTGTINRENYGINFNQALETGGVMLGKDV | |
| KFEASAEFSISE | |
| SEQ ID NO: 21 | |
| - tnpC | |
| MDKQVRNTTEIVRLAKQKSKKTREKVDKAISKFSIEGKVINFNSIAKEANVSK | |
| SWLYKEHDIRQRIESLRERQITANVVSKPKKSSRSEEILIKTLKRRVMELEKEN | |
| KKLQNQIQKLYGDLYNKE | |
| SEQ ID NO: 22 | |
| - UreD | |
| MNVNVEDNAKVTLTSQGATKIYKTPSNHVEQYQTFNLKDNAYLEYVADPIIA | |
| YENAKFYQHNTFNLNNSSSLFYTDILTPGYSKTGEAFKYQYMHLINEIYIEDEL | |
| VTYDNLLLNPNKQSINEIGYMEHYSHYGSAYFIHEDVNQKLIDSVYETISSYSN | |
| TFDCRVAISQLPTHGFAVRIFAYRTQIIEKILGTIQSYIAENIYDRKLDFLRKY | |
| SEQ ID NO: 23 | |
| - NMWN_2109 | |
| MKLKSFVTATLALGLLSTVGAALPSHEASADSNNGYKELTMDGKHTVPYTIS | |
| VDGITALHRTYFVFPENKKVLYQEIDSKVKNELASQRGVTTEKINNAQTATYT | |
| LTLNDGNKKVVNLKKNDDAKNSIDPSTIKQIQIVVK | |
| SEQ ID NO: 24 | |
| - LacB | |
| MKIALGCDHIVTDTKMRVSEFLKSKGHEVIDVGTYDFTRTHYPIFGKKVGEQ | |
| VVSGNADLGVCICGTGVGINNAVNKVPGVRSALVRDMTSALYAKEELNANV | |
| IGFGGRIIGELLMCDIIDAFINAEYKATEENKKLIAKIKHLETSNADQADPHFFD | |
| EFLEKWDRGEYH | |
| SEQ ID NO: 25 | |
| - SA1633 | |
| MNTKFLGKTLVASALVLTTLGTGLHSSYLGLNTNKVVKTAKAEEKMTNGQL | |
| WKKVKDSLIDSNIISGNENEEITVTYVNKTGYSSSVSAYGNNNDDFSSTPSNFS | |
| KLKEIDLKKDNVPSDDFNTTVSGEDSWKTLTSKLKEKGLVTDGQTVTIHCND | |
| KSDNTKSSVSGKVGADLTSGNGTTFKKRFIDKITID | |
| SEQ ID NO: 26 | |
| - NWMN_1623 | |
| MTNQDNNHQLNHRIYHFEKIYKAIKHVIVFIFMIFIAIVAIAVIAMSLYFHHLTK | |
| TSDSLSDDALIKKVRQIPGDELLDHNNKNLLYEYNHSQNSLIIGPKTSSPNVIK | |
| ALTSSEDTLFYKHDGILPKAILRAMIQDIFNTDQSSGGSTITQQLVKNQVLTNE | |
| KTYSRKANELRLAIRLEHLLSKDEIIYTYLNIVPFGRDYNGANISGIASASYSLF | |
| GIPPKDLSIAQSAYLIGLLQSPYGYTPYEKDGTLKSDKDLKYSIQRQHYVLKR | |
| MLIEDQITEKEYNDALKYDIKSHLLNRKKR | |
| SEQ ID NO: 27 | |
| - tnp | |
| MLSFTRMSVRGINKVKRELGFVLMALNIRKIAAQRAVHYKIHIKKADFYQIIN | |
| RNQLFTLPKNLMSQPPS | |
| SEQ ID NO: 28 | |
| - NWNM_0254 | |
| MANLQKYIEYSREVQQARENNQPIVALESTIISHGMPYPQNVEMATTVEQIIR | |
| NNGAIPATIAIIDGKIKIGLESEDLEILATSKDVAKVSRRDLAEVVAMKCVGAT | |
| TVATTMICAAMAGIQFFVTGGIGGVHKGAEHTMDISADLEELSKTNVTVICA | |
| GAKSILDLPKTMEYLETKGVPVIGYQTNELPAFFTRESGVKLTSSVETPERLAD | |
| IHLTKQQLNLEGGIVVANPIPYEHALSKAYIEAIINEAVVEAENQGIKGKDATP | |
| FLLGKIVEKTNGKSLAANIKLVENNAALGAKIAVAVNKLL | |
| SEQ ID NO: 29 | |
| - NMWN_0257 | |
| MTSFIYKILYVVKINAYTYDIMTEDIMILSILLIFFCIRLVSLKISINHSKQLKAD | |
| GAVEYGVKNSKFLAITHVLIYVLAGVEAFINKDTFSFANGIGLVILIFAYIMLF | |
| MVIKTLGGIWTLKLFILPNHPIIKSGLYKITKHPNYFLNIIPELIGVLLLTHATYT | |
| TILLVPYAYFLYVRIKQEEKLMNI | |
| SEQ ID NO: 30 | |
| - SbnB | |
| MNREMLYLNRSDIEQAGGNHSQVYVDALTEALTAHAHNDFVQPLKPYLRQD | |
| PENGHIADRIIAMPSHIGGEHAISGIKWIGSKHDNPSKRNMERASGVIILNDPET | |
| NYPIAVMEASLISSMRTAAVSVIAAKHLAKKGFKDLTIIGCGLIGDKQLQSML | |
| EQFDHIKRVFVYDQFSEACARFVDRWQQQRPEINFIATENAKEAVSNGEVVIT | |
| CTVTDQPYIEYDWLQKGAFISNISIMDVHKEVFIKADKVVVDDWSQCNREKK | |
| TINQLVLEGKFSKEALHAELGQLVTGDIPGREDDDEIILLNPMGMAIEDISSAY | |
| FIYQQAQQQNIGTTLNLY | |
| SEQ ID NO: 31 | |
| - MW0751 | |
| MAIKKKDRIIGVKELEIPQELKLVPNWVLWRAEWNEKQQNYGKVPYSINGYR | |
| ASTTNKKTWCDFESVSIEYEVDEQYSGIGFVLSDGNNFVCLDIDNAIDEKGQI | |
| NSELALKMMRLTYCEKSPSGTGLHCFFKGKLPDNRKKKRTDLDIELYDSARF | |
| MTVTGCTIGQNDICDNQEVLNTLIDEYFKENLPVNDVVREESNTNMQLSDEDI | |
| INIMMKSKQKDKIKDLLQGTYESYFDSSSEAVQSLLHYLAFYTGKNKQQMER | |
| IFLNYNNLTDKWESKRGNTTWGQLELDKAIKNQKTVYTKSIDEFNVIQQGSK | |
| DVKQLLNQLGHEERTKMEEKWIEGGKRGRKPTTISPIKCAYILNEHLTFILFDD | |
| EENTKLAMYQFDEGIYTQNTTIIKRVISYLEPKHNSNKADEVIYHLTNMVDIK | |
| EKTNSPYLIPVKNGVFNRKTKQLESFTPDCIFTTKIDTSYVRQDIVPEINGWNID | |
| RWIEEIACNDNQVVKLLWQVINDSMNGNYTRKKAIFLVGNGNNGKGTFQEL | |
| LSNVIGYSNIASLKVNEFDERFKLSVLEGKTAVIGDDVPVGVYVDDSSNFKSV | |
| VTGDPVLVEFKNKPLYRATFKCTVIQSTNGMPKFKDKTGGTLRRLLIVPFNAN | |
| FNGIKENFKIKEDYIKNPQVLEYVLYKAINLDFETFDIPDASEKMLEVFKEDND | |
| PVYGFKVNMFDQWTIRKVPKYIVYAFYKEYCDENGYNALSSNKFYKQFEHY | |
| LENYWKTDAQRRYDNEELAKRIYNFNDNRNYIEPIESGKNYKSYEKVKLKAI | |
| SEQ ID NO: 32 | |
| - SA0193 | |
| MRKVLNIFTIAFKSILKNKGRNIFTMIGIIIGISSVITIMSLGNGFKKTAADQFSD | |
| AGAGKQEALISFTFKVDEKIKKYPFNQRDIELVNQVDGVLDAKLKENKEEGIE | |
| ATITNVQKKSDIFIIKKQNLHSFKVGRGFDKEDNELRKKIVVINDQVAKTVFN | |
| NNAIGKSLYIEGQGFEVIGITDKLYSDSSTVIMPENTFNYYMGHLHQGLPTLQI | |
| IIEDGYNKKTVVKKVESLLNKKGSGSVLGEYTYTDTEEIIKSIDKIFDSITYFVA | |
| AVAGISLFIAGIGVMNVMYISVAERTEEIAIRRAFGAKSRDIELQFLIESILICVT | |
| SGFIGLILGVVFATIIDVLTPDYIKSVVSLSSVIIAVSVSILIGLLFGWIPARAASK | |
| KELIDIIK | |
| SEQ ID NO: 33 | |
| - NMWN_1106 | |
| MLSFTRMSVRGINKVKRELGFVLMALNIRKIAAQRAVHYKIHIKKADFYQIIN | |
| RNQLFTLPKNLMSQPPS | |
| SEQ ID NO: 34 | |
| - EsxA (NMWN_0219) | |
| MAMIKMSPEEIRAKSQSYGQGSDQIRQILSDLTRAQGEIAANWEGQAFSRFEE | |
| QFQQLSPKVEKFAQLLEEIKQQLNSTADAVQEQDQQLSNNFGLQ | |
| SEQ ID NO: 35 | |
| - EsxA-V5-313 fusion | |
| ggtacCGCTAGCCGCGCCGCCACCATGGATGCAATGAAGAGAGGGCTCTGCT | |
| GTGTGCTGCTGCTGTGTGGAGCAGTCTTCGTTTCGCCCAGCCAGGAAATCC | |
| ATGCCCGATTCAGAAGAGGATCGAAGCTTGCCATGATCAAGATGAGCCCC | |
| GAGGAAATCCGGGCCAAGAGCCAGAGCTACGGCCAGGGCAGCGACCAGA | |
| TCCGGCAGATCCTGAGCGACCTGACCAGAGCCCAGGGCGAGATCGCCGCC | |
| AATTGGGAGGGCCAGGCCTTCAGCAGATTCGAGGAACAGTTTCAGCAGCT | |
| GAGCCCCAAGGTGGAGAAGTTCGCCCAGCTGCTGGAAGAGATCAAGCAG | |
| CAGCTGAACAGCACCGCCGACGCCGTGCAGGAACAGGATCAGCAGCTGTC | |
| CAACAACTTCGGGCTGCAGGGATCCGGGCCCGGGGCTTCAGGTAAGCCTA | |
| TCCCTAACCCTCTCCTCGGTCTCGATTCTACGCGGACCGGACCTGGATCAA | |
| AGAAGCAGGGCGACGCCGACGTGTGTGGCGAGGTGGCCTACATCCAGAG | |
| CGTGGTGTCCGACTGTCACGTGCCCACCGCCGAGCTGAGAACCCTGCTGG | |
| AAATCCGGAAGCTGTTCCTGGAAATCCAGAAACTGAAGGTGGAGCTGCAG | |
| GGCCTGAGCAAAGAGTGATGAGCggccgctcg | |
| SEQ ID NO: 36 | |
| - BitC.V5.313 | |
| GgtacCGCTAGCCGCGCCGCCACCATGGATGCAATGAAGAGAGGGCTCTGC | |
| TGTGTGCTGCTGCTGTGTGGAGCAGTCTTCGTTTCGCCCAGCCAGGAAATC | |
| CATGCCCGATTCAGAAGAGGATCGAAGCTTGCCAACACCCGGCACAGCGA | |
| GAGCGACAAGAACGTGCTGACCGTGTACAGCCCCTACCAGAGCAACCTGA | |
| TCCGGCCCATCCTGAACGAGTTCGAGAAGCAGGAACACGTCAAGATCGAG | |
| ATCAAGCACGGCAGCACACAGGTGCTGCTGAGCAACCTGCACAACGAGG | |
| ACTTCAGCGAGCGGGGCGACGTGTTCATGGGCGGCGTGCTGAGCGAGACA | |
| ATCGACCACCCCGAGGACTTCGTGCCATACCAGGACACCAGCGTGACCCA | |
| GCAGCTGGAAGATTACCGGTCCAACAACAAATACGTGACCAGCTTCCTGC | |
| TGATGCCCACCGTGATCGTGGTCAACAGCGACCTGCAGGGCGACATCAAG | |
| ATCCGGGGCTACCAGGACCTGCTGCAGCCTATCCTGAAGGGCAAGATCGC | |
| CTACAGCAACCCCAACACCACCACCACCGGCTACCAGCACATGCGGGCCA | |
| TCTACAGCATGCACCACCGGGTGTCCGACGTGCACCAGTTCCAGAACCAC | |
| GCCATGCAGCTGAGCAAGACCAGCAAAGTGATCGAGGACGTGGCCAAGG | |
| GCAAGTACTACGCCGGCCTGAGCTACGAGCAGGACGCCCGGACCTGGAA | |
| GAACAAGGGCTACCCCGTGTCCATCGTGTACCCCATCGAGGGCACCATGC | |
| TGAACGTGGACGGAATCGCCCTGGTCAAGAACGCCCACCCCCACCCCAAG | |
| CGGAAGAAACTGGTGCAGTACCTGACCAGCAGAAGCGTGCAGCAGCGGC | |
| TGGTGGCCGAGTTCGACGCCAAGAGCATCCGGAAGGACGTGTCCGAGCAG | |
| AGCGACCAGAGCATCGAGAACCTGAAGAACATCCCCCTGATCCCCAAGAG | |
| CAAGCTGCCCGACATCCCCCACCACAAGTTTCTGGAAATGATCCAGGGAT | |
| CCGGGCCCGGGGCTTCAGGTAAGCCTATCCCTAACCCTCTCCTCGGTCTCG | |
| ATTCTACGCGGACCGGACCTGGATCAAAGAAGCAGGGCGACGCCGACGT | |
| GTGTGGCGAGGTGGCCTACATCCAGAGCGTGGTGTCCGACTGTCACGTGC | |
| CCACCGCCGAGCTGAGAACCCTGCTGGAAATCCGGAAGCTGTTCCTGGAA | |
| ATCCAGAAACTGAAGGTGGAGCTGCAGGGCCTGAGCAAAGAGTGATGAG | |
| Cggccgctc | |
| SEQ ID NO: 37 | |
| - EsxA.rLuc | |
| gtaccgaattcaccGCCGCCAaCATGAAGCTTGCCATGATCAAGATGAGCCCCGAG | |
| GAAATCCGGGCCAAGAGCCAGAGCTACGGCCAGGGCAGCGACCAGATCC | |
| GGCAGATCCTGAGCGACCTGACCAGAGCCCAGGGCGAGATCGCCGCCAAT | |
| TGGGAGGGCCAGGCCTTCAGCAGATTCGAGGAACAGTTTCAGCAGCTGAG | |
| CCCCAAGGTGGAGAAGTTCGCCCAGCTGCTGGAAGAGATCAAGCAGCAG | |
| CTGAACAGCACCGCCGACGCCGTGCAGGAACAGGATCAGCAGCTGTCCAA | |
| CAACTTCGGGCTGCAGGGATCCGGGCCCGGGGCCATGGGTTCGAAGGTGT | |
| ACGACCCCGAGCAACgcaaacgcatgatcactgggcctcagtggtgggctcgctgcaagcaaatgaacgt | |
| gctggactccttcatcaactactatgattccgagaagcacgccgagaacgccgtgatCtttctgcatggtaacgctacctcc | |
| agctacctgtggaggcacgtcgtgcctcacatcgagcccgtggctagatgcatcatccctgatctgatcggaatgggtaagt | |
| ccggcaagagcgggaatggctcatatcgcctcctggatcactacaagtacctcaccgcttggttcgagctgctgaaccttcc | |
| aaagaaaatcatctttgtgggccacgactggggggctgctctggcctttcactacgcctacgagcaccaagacaggatcaa | |
| ggccatcgtccatatggagagtgtcgtggacgtgatcgagtcctgggacgagtggcctgacatcgaggaggatatcgccc | |
| tgatcaagagcgaagagggcgagaaaatggtgcttgagaataacttcttcgtcgagaccgtgctcccaagcaagatcatgc | |
| ggaaactggagcctgaggagttcgctgcctacctggagccattcaaggagaagggcgaggttagacggcctaccctctc | |
| ctggcctcgcgagatccctctcgttaagggaggcaagcccgacgtcgtccagattgtccgcaactacaacgcctaccttcg | |
| ggccagcgacgatctgcctaagctgttcatcgagtccgaccctgggttcttttccaacgctattgtcgagggagctaagaag | |
| ttccctaacaccgagttcgtgaaggtgaagggcctccacttcctccaggaggacgctccagatgaaatgggtaagtacatc | |
| aagagcttcgtggagCGCGTGCTGAAGAACGAGCAGtaatTCTAGATTTTTATAGC | |
| SEQ ID NO: 38 | |
| - BitC.rLuc | |
| gtaccgaattcaccGCCGCCAaCATGAAGCTTGCCAACACCCGGCACAGCGAGAG | |
| CGACAAGAACGTGCTGACCGTGTACAGCCCCTACCAGAGCAACCTGATCC | |
| GGCCCATCCTGAACGAGTTCGAGAAGCAGGAACACGTCAAGATCGAGATC | |
| AAGCACGGCAGCACACAGGTGCTGCTGAGCAACCTGCACAACGAGGACTT | |
| CAGCGAGCGGGGCGACGTGTTCATGGGCGGCGTGCTGAGCGAGACAATC | |
| GACCACCCCGAGGACTTCGTGCCATACCAGGACACCAGCGTGACCCAGCA | |
| GCTGGAAGATTACCGGTCCAACAACAAATACGTGACCAGCTTCCTGCTGA | |
| TGCCCACCGTGATCGTGGTCAACAGCGACCTGCAGGGCGACATCAAGATC | |
| CGGGGCTACCAGGACCTGCTGCAGCCTATCCTGAAGGGCAAGATCGCCTA | |
| CAGCAACCCCAACACCACCACCACCGGCTACCAGCACATGCGGGCCATCT | |
| ACAGCATGCACCACCGGGTGTCCGACGTGCACCAGTTCCAGAACCACGCC | |
| ATGCAGCTGAGCAAGACCAGCAAAGTGATCGAGGACGTGGCCAAGGGCA | |
| AGTACTACGCCGGCCTGAGCTACGAGCAGGACGCCCGGACCTGGAAGAA | |
| CAAGGGCTACCCCGTGTCCATCGTGTACCCCATCGAGGGCACCATGCTGA | |
| ACGTGGACGGAATCGCCCTGGTCAAGAACGCCCACCCCCACCCCAAGCGG | |
| AAGAAACTGGTGCAGTACCTGACCAGCAGAAGCGTGCAGCAGCGGCTGG | |
| TGGCCGAGTTCGACGCCAAGAGCATCCGGAAGGACGTGTCCGAGCAGAG | |
| CGACCAGAGCATCGAGAACCTGAAGAACATCCCCCTGATCCCCAAGAGCA | |
| AGCTGCCCGACATCCCCCACCACAAGTTTCTGGAAATGATCCAGGGATCC | |
| GGGCCCGGGGCCATGGGTTCGAAGGTGTACGACCCCGAGCAACgcaaacgcat | |
| gatcactgggcctcagtggtgggctcgctgcaagcaaatgaacgtgctggactccttcatcaactactatgattccgagaag | |
| cacgccgagaacgccgtgatCtttctgcatggtaacgctacctccagctacctgtggaggcacgtcgtgcctcacatcgag | |
| cccgtggctagatgcatcatccctgatctgatcggaatgggtaagtccggcaagagcgggaatggctcatatcgcctcctg | |
| gatcactacaagtacctcaccgcttggttcgagctgctgaaccttccaaagaaaatcatctttgtgggccacgactgggggg | |
| ctgctctggcctttcactacgcctacgagcaccaagacaggatcaaggccatcgtccatatggagagtgtcgtggacgtga | |
| tcgagtcctgggacgagtggcctgacatcgaggaggatatcgccctgatcaagagcgaagagggcgagaaaatggtgct | |
| tgagaataacttcttcgtcgagaccgtgctcccaagcaagatcatgeggaaactggagcctgaggagttcgctgcctacct | |
| ggagccattcaaggagaagggcgaggttagacggcctaccctctcctggcctcgcgagatccctctcgttaagggaggc | |
| aagcccgacgtcgtccagattgtccgcaactacaacgcctaccttcgggccagcgacgatctgcctaagctgttcatcgag | |
| tccgaccctgggttcttttccaacgctattgtcgagggagctaagaagttccctaacaccgagttcgtgaaggtgaagggcc | |
| tccacttcctccaggaggacgctccagatgaaatgggtaagtacatcaagagcttcgtggagCGCGTGCTGAAG | |
| AACGAGCAGtaatTCTAGATTTTTATAGC | |
| SEQ ID NO: 39 - pMono2.BitC.313 | |
| LOCUS | B9_pMono2_BitC-313 4596 bp DNA circular 24-SEP-2012 |
| FEATURES | Location/Qualifiers |
| gene | 2 . . . 936 |
| | /note = “KanR” |
| gene | 954 . . . 1650 |
| | /note = “puc19_ori” |
| terminator | 1756 . . . 1799 |
| | /note = “rrnB_T1 transcriptional terminator” |
| terminator | 1931 . . . 1958 |
| | /note = “rrnB_T2 transcriptional terminator” |
| misc_recomb | 2008 . . . 2107 |
| | /note = “attL1” |
| misc_feature | 2134 . . . 2330 |
| | /note = “CMVIE_nosplice_TO” |
| PCR_primer | 1984 . . . 2005 |
| | /dnas_title = “AGTTAGTTACTTAAGCTCGGGC” |
| | /pair = “” |
| | /primer = “AGTTAGTTACTTAAGCTCGGGC” |
| | /current = 0 |
| misc_feature | 1984 . . . 2814 |
| | /note = “sequenced 09.12.09” |
| misc_feature | 2331 . . . 2806 |
| | /note = “CMVIE_TO” |
| PCR_primer | 2389 . . . 2410 |
| | /dnas_title = “GCCCAGTACATGACCTTATGGG” |
| | /pair = “” |
| | /primer = “GCCCAGTACATGACCTTATGGG” |
| | /current = 0 |
| PCR_primer | complement(2448 . . . 2467) |
| | /dnas_title = “GCATCACCATGGTAATAGCG” |
| | /pair = “” |
| | /primer = “GCATCACCATGGTAATAGCG” |
| | /current = 0 |
| TATA_signal | 2650 . . . 2656 |
| | /note = “” |
| misc_feature | 2563 . . . 2563 |
| | /note = “single nt change (A-->C) based on sequencing |
| | result 25.11.09” |
| PCR_primer | 2572 . . . 2591 |
| | /dnas_title = “CAACGGGACTTTCCAAAATG” |
| | /pair = “CMVF Sequencing primer (Geneservice)” |
| | /primer = “CAACGGGACTTTCCAAAATG” |
| | /current = 0 |
| protein_bind | 2666 . . . 2705 |
| | /note = “TOx2” |
| misc_feature | 2802 . . . 2811 |
| | /note = “KlenowPmeISiteDestruction” |
| misc_feature | 2813 . . . 2813 |
| | /note = “modified by linker ligation to remove HindIII site” |
| misc_feature | 2842 . . . 2933 |
| | /note = “TPAleader” |
| PCR_primer | 2714 . . . 2734 |
| | /dnas_title = “GAGCTCGTTTAGTGAACCGTC” |
| | /pair = “CMVF (geneservice)” |
| | /primer = “GAGCTCGTTTAGTGAACCGTC” |
| | /current = 0 |
| misc_feature | 2941 . . . 3858 |
| | /note = “sport_top1” |
| misc_feature | 2941 . . . 3858 |
| | /note = “BitC from 2452” |
| misc_feature | 3939 . . . 4103 |
| | /note = “IMX313_tag” |
| misc_feature | 3927 . . . 3938 |
| | /note = “linker” |
| misc_feature | 3879 . . . 3926 |
| | /note = “V5_epitope_tag GKPIPNPLLGLDST” |
| misc_feature | 3868 . . . 3878 |
| | /note = “linker” |
| misc_feature | 3859 . . . 3867 |
| | /note = “3primerpolylinker” |
| misc_feature | 3859 . . . 3867 |
| | /note = “sport_top1” |
| misc_feature | 4188 . . . 4412 |
| | /note = “BGH_polyA” |
| misc_feature | complement(4170 . . . 4194) |
| | /note = “antigenR primer” |
| misc_feature | 4192 . . . 4213 |
| | /note = “” |
| PCR_primer | complement(4182 . . . 4199) |
| | /dnas_title = “TAGAAGGCACAGTCGAGG” |
| | /pair = “bGHR primer sequence” |
| | /primer = “TAGAAGGCACAGTCGAGG” |
| | /current = 1 |
| misc_recomb | 4475 . . . 4574 |
| | /note = “attL2” |
| misc_feature | 2790 . . . 3477 |
| | /note = “Sequence confirmed with primer CMVF (Geneservice)” |
| source | 1 . . . 4596 |
| | /dnas title = “B9_pMono2_BitC-313” |
| ORIGIN | |
| 1 CGTGTCTCAA AATCTCTGAT GTTACATTGC ACAAGATAAA AATATATCAT CATGAACAAT | |
| 61 AAAACTGTCT GCTTACATAA ACAGTAATAC AAGGGGTGTT ATGAGCCATA TTCAACGGGA | |
| 121 AACGTCGAGG CCGCGATTAA ATTCCAACAT GGATGCTGAT TTATATGGGT ATAAATGGGC | |
| 181 TCGCGATAAT GTCGGGCAAT CAGGTGCGAC AATCTATCGC TTGTATGGGA AGCCCGATGC | |
| 241 GCCAGAGTTG TTTCTGAAAC ATGGCAAAGG TAGCGTTGCC AATGATGTTA CAGATGAGAT | |
| 301 GGTCAGACTA AACTGGCTGA CGGAATTTAT GCCTCTTCCG ACCATCAAGC ATTTTATCCG | |
| 361 TACTCCTGGT GATGCATGGT TACTCACCAC TGCGATCCCC GGAAAAACAG CATTCCAGGT | |
| 421 ATTAGAAGAA TATCCTGATT CAGGTGAAAA TATTGTTGAT GCGCTGGCAG TGTTCCTGCG | |
| 481 CCGGTTGCAT TCGATTCCTG TTTGTAATTG TCCTTTTAAC AGCGATCGCG TATTTCGTCT | |
| 541 CGCTCAGGCG CAATCACGAA TGAATAACGG TTTGGTTGAT GCGAGTGATT TTGATGACGA | |
| 601 GCGTAATGGC TGGCCTGTTG AACAAGTCTG GAAAGAAATG CATAAACTTT TGCCATTCTC | |
| 661 ACCGGATTCA GTCGTCACTC ATGGTGATTT CTCACTTGAT AACCTTATTT TTGACGAGGG | |
| 721 GAAATTAATA GGTTGTATTG ATGTTGGACG AGTCGGAATC GCAGACCGAT ACCAGGATCT | |
| 781 TGCCATCCTA TGGAACTGCC TCGGTGAGTT TTCTCCTTCA TTACAGAAAC GGCTTTTTCA | |
| 841 AAAATATGGT ATTGATAATC CTGATATGAA TAAATTGCAG TTTCATTTGA TGCTCGATGA | |
| 901 GTTTTTCTAA TCAGAATTGG TTAATTGGTT GTAACATTAT TCAGATTGGG CCCCGTTCCA | |
| 961 CTGAGCGTCA GACCCCGTAG AAAAGATCAA AGGATCTTCT TGAGATCCTT TTTTTCTGCG | |
| 1021 CGTAATCTGC TGCTTGCAAA CAAAAAAACC ACCGCTACCA GCGGTGGTTT GTTTGCCGGA | |
| 1081 TCAAGAGCTA CCAACTCTTT TTCCGAAGGT AACTGGCTTC AGCAGAGCGC AGATACCAAA | |
| 1141 TACTGTTCTT CTAGTGTAGC CGTAGTTAGG CCACCACTTC AAGAACTCTG TAGCACCGCC | |
| 1201 TACATACCTC GCTCTGCTAA TCCTGTTACC AGTGGCTGCT GCCAGTGGCG ATAAGTCGTG | |
| 1261 TCTTACCGGG TTGGACTCAA GACGATAGTT ACCGGATAAG GCGCAGCGGT CGGGCTGAAC | |
| 1321 GGGGGGTTCG TGCACACAGC CCAGCTTGGA GCGAACGACC TACACCGAAC TGAGATACCT | |
| 1381 ACAGCGTGAG CTATGAGAAA GCGCCACGCT TCCCGAAGGG AGAAAGGCGG ACAGGTATCC | |
| 1441 GGTAAGCGGC AGGGTCGGAA CAGGAGAGCG CACGAGGGAG CTTCCAGGGG GAAACGCCTG | |
| 1501 GTATCTTTAT AGTCCTGTCG GGTTTCGCCA CCTCTGACTT GAGCGTCGAT TTTTGTGATG | |
| 1561 CTCGTCAGGG GGGCGGAGCC TATGGAAAAA CGCCAGCAAC GCGGCCTTTT TACGGTTCCT | |
| 1621 GGCCTTTTGC TGGCCTTTTG CTCACATGTT CTTTCCTGCG TTATCCCCTG ATTCTGTGGA | |
| 1681 TAACCGTATT ACCGCTAGCA TGGATCTCGG GGACGTCTAA CTACTAAGCG AGAGTAGGGA | |
| 1741 ACTGCCAGGC ATCAAATAAA ACGAAAGGCT CAGTCGGAAG ACTGGGCCTT TCGTTTTATC | |
| 1801 TGTTGTTTGT CGGTGAACGC TCTCCTGAGT AGGACAAATC CGCCGGGAGC GGATTTGAAC | |
| 1861 GTTGTGAAGC AACGGCCCGG AGGGTGGCGG GCAGGACGCC CGCCATAAAC TGCCAGGCAT | |
| 1921 CAAACTAAGC AGAAGGCCAT CCTGACGGAT GGCCTTTTTG CGTTTCTACA AACTCTTCCT | |
| 1981 GTTAGTTAGT TACTTAAGCT CGGGCCCCAA ATAATGATTT TATTTTGACT GATAGTGACC | |
| 2041 TGTTCGTTGC AACAAATTGA TAAGCAATGC TTTTTTATAA TGCCAACTTT GTACAAAAAA | |
| 2101 GCAGGCTCCA CCATGGGAAC CAATTCAgTC GAGCCTTTCA CTCATTAGAT GCATGTCGTT | |
| 2161 ACATAACTTA CGGTAAATGG CCCGCCTGGC TGACCGCCCA ACGACCCCCG CCCATTGACG | |
| 2221 TCAATAATGA CGTATGTTCC CATAGTAACG CCAATAGGGA CTTTCCATTG ACGTCAATGG | |
| 2281 GTGGAGTATT TACGGTAAAC TGCCCACTTG GCAGTACATC AAGTGTATCA TATGCCAAGT | |
| 2341 ACGCCCCCTA TTGACGTCAA TGACGGTAAA TGGCCCGCCT GGCATTATGC CCAGTACATG | |
| 2401 ACCTTATGGG ACTTTCCTAC TTGGCAGTAC ATCTACGTAT TAGTCATCGC TATTACCATG | |
| 2461 GTGATGCGGT TTTGGCAGTA CATCAATGGG CGTGGATAGC GGTTTGACTC ACGGGGATTT | |
| 2521 CCAAGTCTCC ACCCCATTGA CGTCAATGGG AGTTTGTTTT GGCACCAAAA TCAACGGGAC | |
| 2581 TTTCCAAAAT GTCGTAACAA CTCCGCCCCA TTGACGCAAA TGGGCGGTAG GCGTGTACGG | |
| 2641 TGGGAGGTCT ATATAAGCAG AGCTCTCCCT ATCAGTGATA GAGATCTCCC TATCAGTGAT | |
| 2701 AGAGATCGTC GACGAGCTCG TTTAGTGAAC CGTCAGATCG CCTGGAGACG CCATCCACGC | |
| 2761 TGTTTTGACC TCCATAGAAG ACACCGGGAC CGATCCAGCC TCCGGTTAAG CTcGgtacCG | |
| 2821 CTAGCCGCGC CGCCACCATG GATGCAATGA AGAGAGGGCT CTGCTGTGTG CTGCTGCTGT | |
| 2881 GTGGAGCAGT CTTCGTTTCG CCCAGCCAGG AAATCCATGC CCGATTCAGA AGAGGATCGA | |
| 2941 AGCTTGCCAA CACCCGGCAC AGCGAGAGCG ACAAGAACGT GCTGACCGTG TACAGCCCCT | |
| 3001 ACCAGAGCAA CCTGATCCGG CCCATCCTGA ACGAGTTCGA GAAGCAGGAA CACGTCAAGA | |
| 3061 TCGAGATCAA GCACGGCAGC ACACAGGTGC TGCTGAGCAA CCTGCACAAC GAGGACTTCA | |
| 3121 GCGAGCGGGG CGACGTGTTC ATGGGCGGCG TGCTGAGCGA GACAATCGAC CACCCCGAGG | |
| 3181 ACTTCGTGCC ATACCAGGAC ACCAGCGTGA CCCAGCAGCT GGAAGATTAC CGGTCCAACA | |
| 3241 ACAAATACGT GACCAGCTTC CTGCTGATGC CCACCGTGAT CGTGGTCAAC AGCGACCTGC | |
| 3301 AGGGCGACAT CAAGATCCGG GGCTACCAGG ACCTGCTGCA GCCTATCCTG AAGGGCAAGA | |
| 3361 TCGCCTACAG CAACCCCAAC ACCACCACCA CCGGCTACCA GCACATGCGG GCCATCTACA | |
| 3421 GCATGCACCA CCGGGTGTCC GACGTGCACC AGTTCCAGAA CCACGCCATG CAGCTGAGCA | |
| 3481 AGACCAGCAA AGTGATCGAG GACGTGGCCA AGGGCAAGTA CTACGCCGGC CTGAGCTACG | |
| 3541 AGCAGGACGC CCGGACCTGG AAGAACAAGG GCTACCCCGT GTCCATCGTG TACCCCATCG | |
| 3601 AGGGCACCAT GCTGAACGTG GACGGAATCG CCCTGGTCAA GAACGCCCAC CCCCACCCCA | |
| 3661 AGCGGAAGAA ACTGGTGCAG TACCTGACCA GCAGAAGCGT GCAGCAGCGG CTGGTGGCCG | |
| 3721 AGTTCGACGC CAAGAGCATC CGGAAGGACG TGTCCGAGCA GAGCGACCAG AGCATCGAGA | |
| 3781 ACCTGAAGAA CATCCCCCTG ATCCCCAAGA GCAAGCTGCC CGACATCCCC CACCACAAGT | |
| 3841 TTCTGGAAAT GATCCAGGGA TCCGGGCCCG GGGCTTCAGG TAAGCCTATC CCTAACCCTC | |
| 3901 TCCTCGGTCT CGATTCTACG CGGACCGGAC CTGGATCAAA GAAGCAGGGC GACGCCGACG | |
| 3961 TGTGTGGCGA GGTGGCCTAC ATCCAGAGCG TGGTGTCCGA CTGTCACGTG CCCACCGCCG | |
| 4021 AGCTGAGAAC CCTGCTGGAA ATCCGGAAGC TGTTCCTGGA AATCCAGAAA CTGAAGGTGG | |
| 4081 AGCTGCAGGG CCTGAGCAAA GAGTGATGAG Cggccgctcg agcatgcatc tagagggccc | |
| 4141 tattctatag tgtcacctaa atgctagagc tcgctgatca gcctcgactg tgccttctag | |
| 4201 ttgccagcca tctgttgttt gcccctcccc cgtgccttcc ttgaccctgg aaggtgccac | |
| 4261 tcccactgtc ctttcctaat aaaatgagga aattgcatcg cattgtctga gtaggtgtca | |
| 4321 ttctattctg gggggtgggg tggggcagga cagcaagggg gaggattggg aagacaatag | |
| 4381 caggcatgct ggggatgcgg tgggctctat ggcttctgag gcggaaagaa ccagctgggg | |
| 4441 ctcgaggggg gatcgatccc gtcGAGATAT CTAGACCCAG CTTTCTTGTA CAAAGTTGGC | |
| 4501 ATTATAAGAA AGCATTGCTT ATCAATTTGT TGCAACGAAC AGGTCACTAT CAGTCAAAAT | |
| 4561 AAAATCATTA TTTGCCATCC AGCTGCAGCT CTGGCC | |
| // | |
| SEQ ID NO: 40 - pMVA.BitC.313 | |
| LOCUS | 2658 6763 bp DNA circular 24-SEP-2012 |
| FEATURES | Location/Qualifiers |
| misc_feature | 246 . . . 1099 |
| | /note = “TKR” |
| misc_feature | 2512 . . . 2775 |
| | /note = “FP4b prom” |
| misc_feature | 2776 . . . 2807 |
| | /note = “FP4b” |
| misc_feature | 2808 . . . 3546 |
| | /note = “GFP” |
| misc_feature | 3591 . . . 4348 |
| | /note = “TKL” |
| misc_feature | 3560 . . . 3594 |
| | /note = “FRT” |
| primer_bind | complement(2868 . . . 2886) |
| | /note = “pEGFPn1 reverse sequencing primer” |
| primer_bind | 986 . . . 1009 |
| | /note = “TKR_fwd seq primer” |
| misc_feature | 4962 . . . 5822 |
| | /note = “AmpR” |
| misc_feature | complement(2460 . . . 2500) |
| | /note = “ssp” |
| misc_feature | 3549 . . . 3554 |
| | /note = “old NotI site in p1864” |
| misc_feature | 3480 . . . 3501 |
| | /note = “EGFP C F primer (Geneservice)” |
| misc_feature | complement(2872 . . . 2893) |
| | /note = “EGFP Nrev primer (Geneservice)” |
| misc_feature | complement(1415 . . . 2332) |
| | /note = “sport_top1” |
| misc_feature | complement(1415 . . . 2332) |
| | /note = “BitC from 2452” |
| misc_feature | complement(1170 . . . 1334) |
| | /note = “IMX313_tag” |
| misc_feature | complement(1335 . . . 1346) |
| | /note = “linker” |
| misc_feature | complement(1347 . . . 1394) |
| | /note = “V5_epitope_tag GKPIPNPLLGLDST” |
| misc_feature | complement(1395 . . . 1405) |
| | /note = “linker” |
| misc_feature | complement(1406 . . . 1414) |
| | /note = “3primerpolylinker” |
| misc_feature | complement(1406 . . . 1414) |
| | /note = “sport_top1” |
| misc_feature | complement(2340 . . . 2431) |
| | /note = “TPAleader” |
| PCR_primer | complement(2556 . . . 2580) |
| | /dnas_title = “AATGGTACTTTAATAGGTGGTAGGA” |
| | /pair = “” |
| | /primer = “AATGGTACTTTAATAGGTGGTAGGA” |
| | /current = 1 |
| misc_feature | 1831 . . . 2501 |
| | /note = “Sequence confirmed with FP4b_R primer |
| | (Geneservice)” |
| source | 1 . . . 6763 |
| | /dnas_title = “2658 MVA-BitC-313” |

```
ORIGIN
    1 gcgcccaata cgcaaaccgc ctctccccgc gcgttggccg attcattaat gcagctggca
    61 cgacaggttt cccgactgga aagcgggcag tgagcgcaac gcaattaatg tgagttagct
  121 cactcattag gcaccccagg ctttacactt tatgcttccg gctcgtatgt tgtgtggaat
  181 tgtgagcgga taacaatttc acacaggaaa cagctatgac catgattacg ccaagcttgc
  241 atgcATCTGG AAACGGGCAT CTCCATTTAA GACTAGATGC CACGGGGTTT AAAATACTAA
  301 TCATGACATT TTGTAGAGCG TAATTACTTA GTAAATCCGC CGTACTAGGT TCATTTCCTC
  361 CTCGTTTGGA TCTCACATCA GAAATTAAAA TAATCTTAGA AGGATGCAGT TGTTTTTTGA
  421 TGGATCGTAG ATATTCCTCA TCAACGAACC GAGTCACTAG AGTCACATCA CGCAATCCAT
  481 TTAAAATAGG ATCATGATGG CGGCCGTCAA TTAGCATCCA TTTGATGATC ACTCCTAAAT
  541 TATAGAAATG ATCTCTCAAA TAACGTATAT GTGTACCGGG AGCAGATCCT ATATACACTA
  601 CGGTGGCACC ATCTAATATA CCGTGTCGCT GTAACTTACT AAGAAAAAAT AATTCTCCTA
  661 GTAATAGTTT TAACTGTCCT TGATACGGTA GTTTTTTTGC GACCTCATTT GCACTTTCTG
  721 GTTCGTAATC TAACTCATTA TCAATTTCCT CAAAATACAT AAACGGTTTA TCTAACGACA
  781 CAACATCCAT TTTTAAGTAT TATATTAAAA TTTAATCAAT GTTTATTTTT AGTTTTTTAG
  841 ATTCAAAATA TAATATTATG AGTCGATGTA ACACTTTCTA CACACCGATT GATACATATC
  901 ATTACCTCCT ATTATTTCTA TCTCGGTTTC CTCACCCAAT CGTTTAGAAA AGGAAGCCTC
  961 CTTAAAGCAT TTCATACACA CAGCAGTTAG TTTTACCACC ATTTCAGATA ATGGAATAAG
1021 ATTCAAAATA TTATTAAACG GTTTACGTTG AAATGTCCCA TCGAGTGCGG CTACTATAAC
1081 TATTTTTCCT TCGTTTGCCA TACAGATCCT ACGTACtcga gataaaaagc ggccttggcc
1141 gaggcggcca cgcgtgcggc cGCTCATCAC TCTTTGCTCA GGCCCTGCAG CTCCACCTTC
1201 AGTTTCTGGA TTTCCAGGAA CAGCTTCCGG ATTTCCAGCA GGGTTCTCAG CTCGGCGGTG
1261 GGCACGTGAC AGTCGGACAC CACGCTCTGG ATGTAGGCCA CCTCGCCACA CACGTCGGCG
1321 TCGCCCTGCT TCTTTGATCC AGGTCCGGTC CGCGTAGAAT CGAGACCGAG GAGAGGGTTA
1381 GGGATAGGCT TACCTGAAGC CCCGGGCCCG GATCCCTGGA TCATTTCCAG AAACTTGTGG
1441 TGGGGGATGT CGGGCAGCTT GCTCTTGGGG ATCAGGGGGA TGTTCTTCAG GTTCTCGATG
1501 CTCTGGTCGC TCTGCTCGGA CACGTCCTTC CGGATGCTCT TGGCGTCGAA CTCGGCCACC
1561 AGCCGCTGCT GCACGCTTCT GCTGGTCAGG TACTGCACCA GTTTCTTCCG CTTGGGGTGG
1621 GGGTGGGCGT TCTTGACCAG GGCGATTCCG TCCACGTTCA GCATGGTGCC CTCGATGGGG
1681 TACACGATGG ACACGGGGTA GCCCTTGTTC TTCCAGGTCC GGGCGTCCTG CTCGTAGCTC
1741 AGGCCGGCGT AGTACTTGCC CTTGGCCACG TCCTCGATCA CTTTGCTGGT CTTGCTCAGC
1801 TGCATGGCGT GGTTCTGGAA CTGGTGCACG TCGGACACCC GGTGGTGCAT GCTGTAGATG
1861 GCCCGCATGT GCTGGTAGCC GGTGGTGGTG GTGTTGGGGT TGCTGTAGGC GATCTTGCCC
1921 TTCAGGATAG GCTGCAGCAG GTCCTGGTAG CCCCGGATCT TGATGTCGCC CTGCAGGTCG
1981 CTGTTGACCA CGATCACGGT GGGCATCAGC AGGAAGCTGG TCACGTATTT GTTGTTGGAC
2041 CGGTAATCTT CCAGCTGCTG GGTCACGCTG GTGTCCTGGT ATGGCACGAA GTCCTCGGGG
2101 TGGTCGATTG TCTCGCTCAG CACGCCGCCC ATGAACACGT CGCCCCGCTC GCTGAAGTCC
2161 TCGTTGTGCA GGTTGCTCAG CAGCACCTGT GTGCTGCCGT GCTTGATCTC GATCTTGACG
2221 TGTTCCTGCT TCTCGAACTC GTTCAGGATG GGCCGGATCA GGTTGCTCTG GTAGGGGCTG
2281 TACACGGTCA GCACGTTCTT GTCGCTCTCG CTGTGCCGGG TGTTGGCAAG CTTCGATCCT
2341 CTTCTGAATC GGGCATGGAT TTCCTGGCTG GGCGAAACGA AGACTGCTCC ACACAGCAGC
2401 AGCACACAGC AGAGCCCTCT CTTCATTGCA TCCATGGTGG CGGCGCGGCT AGCggtaccT
2461 TATTTATATT CCAAAAAAAA AAAATAAAAT TTCAATTTTT acaaaaaGAA TTcaTCCACT
2521 TTGGATAAGA AATCTGCATG ATAAATATAT TGATATCCTA CCACCTATTA AAGTACCATT
2581 ATCTAATAGC AATAAGATAG ATAAACAAAT GTTTTTTGAT GAAGTTATTA CGTGGATAAA
2641 TATATATCTT CAGGAAAAGG GTATTATGTT ACCAGATGAT ATAAGAGAAC TCAGAGATGC
2701 TATTATTCCT TAACTAGTTA CGTCTCTTTA GGTACTTATT TTGATACGTT ACAAGTAAAA
2761 AACTATCAAA TATAAATGGA ATCTGATTCT AATATAGCGA TTGAAGAgga TCCACCGGTC
2821 GCCACCATGG TGAGCAAGGG CGAGGAGCTG TTCACCGGGG TGGTGCCCAT CCTGGTCGAG
2881 CTGGACGGCG ACGTAAACGG CCACAAGTTC AGCGTGTCCG GCGAGGGCGA GGGCGATGCC
2941 ACCTACGGCA AGCTGACCCT GAAGTTCATC TGCACCACCG GCAAGCTGCC CGTGCCCTGG
3001 CCCACCCTCG TGACCACCCT GACCTACGGC GTGCAGTGCT TCAGCCGCTA CCCCGACCAC
3061 ATGAAGCAGC ACGACTTCTT CAAGTCCGCC ATGCCCGAAG GCTACGTCCA GGAGCGCACC
3121 ATCTTCTTCA AGGACGACGG CAACTACAAG ACCCGCGCCG AGGTGAAGTT CGAGGGCGAC
3181 ACCCTGGTGA ACCGCATCGA GCTGAAGGGC ATCGACTTCA AGGAGGACGG CAACATCCTG
3241 GGGCACAAGC TGGAGTACAA CTACAACAGC CACAACGTCT ATATCATGGC CGACAAGCAG
3301 AAGAACGGCA TCAAGGTGAA CTTCAAGATC CGCCACAACA TCGAGGACGG CAGCGTGCAG
3361 CTCGCCGACC ACTACCAGCA GAACACCCCC ATCGGCGACG GCCCCGTGCT GCTGCCCGAC
3421 AACCACTACC TGAGCACCCA GTCCGCCCTG AGCAAAGACC CCAACGAGAA GCGCGATCAC
3481 ATGGTCCTGC TGGAGTTCGT GACCGCCGCC GGGATCACTC TCGGCATGGA CGAGCTGTAC
3541 AAGTAAAGCG GccGGccgcg aagttcctat actttctaga gaataggaac TTCAACAATG
3601 TCTGGAAAGA ACTGTCCTTC ATCGATACCT ATCACGGAGA AATCTGTAAT TGATTCCAAG
3661 AcATCACATA GTTTAGTTGC TTCCAATGCT TCAAAATTAT TCTTATCATG CGTCCATAGT
3721 CCCGTTCCGT ATCTATTATC GTTAGAATAT TTTATAGTCA CGCATTTATA TTGAGCTATT
3781 TGATAACGTC TAACTCGTCT AATTAATTCT GTACTTTTAC CTGAAAACAT GGGGCCGATT
3841 ATCAACTGAA TATGTCCGCC GTTCATGATG ACAATAAAGA ATTAATTATT GTTCACTTTA
3901 TTCGACTTTA ATATATCCAT CACGTTAGAA AATGCGATAT cGCGACGAGG ATCTATGTAT
3961 CTAACAGGAT CTATTGCGGT GGTAGCTAGA GctGATTCTT TTTTGAATCG CATCAAACTA
4021 ATCACAAAGT CGAACAAATA TCCTTTATTA AGTTTGACCC TTCCATCTGT AACAATAGGG
4081 ACCTTGTTAA ACAGTTTTTT AAAATCTTGA gAGTCTGTGA ATTTTGTCAA TTGTCTGTAT
4141 TCCTCTGAAA GAGATTCATA ACAATGACCC ACGGCTTCTA ATTTATTTTT TGATTGGATC
4201 AATAATAATA ACAGAAAGTC TAGATATTGA GTGATTTGCA ATATATCAGA TAATGAAGAT
4261 TCATCATCTT GACTAGCCAA ATACTTAAAA AATGAATCAT CATCTGCGAA GAACATCGTT
4321 AAGAGATACT GGTTGTGATC CATTTATgag ctcgcgaaag cttggcactg gccgtcgttt
4381 tacaacgtcg tgactgggaa aaccctggcg ttacccaact taatcgcctt gcagcacatc
4441 cccctttcgc cagctggcgt aatagcgaag aggcccgcac cgatcgccct tcccaacagt
4501 tgcgcagcct gaatggcgaa tggcgcctga tgcggtattt tctccttacg catctgtgcg
4561 gtatttcaca ccgcatatgg tgcactctca gtacaatctg ctctgatgcc gcatagttaa
4621 gccagccccg acacccgcca acacccgctg acgcgccctg acgggcttgt ctgctcccgg
4681 catccgctta cagacaagct gtgaccgtct ccgggagctg catgtgtcag aggttttcac
4741 cgtcatcacc gaaacgcgcg agacgaaagg gcctcgtgat acgcctattt ttataggtta
4801 atgtcatgat aataatggtt tcttagacgt caggtggcac ttttcgggga aatgtgcgcg
4861 gaacccctat ttgtttattt ttctaaatac attcaaatat gtatccgctc atgagacaat
4921 aaccctgata aatgcttcaa taatattgaa aaaggaagag tatgagtatt caacatttcc
4981 gtgtcgccct tattcccttt tttgcggcat tttgccttcc tgtttttgct cacccagaaa
5041 cgctggtgaa agtaaaagat gctgaagatc agttgggtgc acgagtgggt tacatcgaac
5101 tggatctcaa cagcggtaag atccttgaga gttttcgccc cgaagaacgt tttccaatga
5161 tgagcacttt taaagttctg ctatgtggcg cggtattatc ccgtattgac gccgggcaag
5221 agcaactcgg tcgccgcata cactattctc agaatgactt ggttgagtac tcaccagtca
5281 cagaaaagca tcttacggat ggcatgacag taagagaatt atgcagtgct gccataacca
5341 tgagtgataa cactgcggcc aacttacttc tgacaacgat cggaggaccg aaggagctaa
5401 ccgctttttt gcacaacatg ggggatcatg taactcgcct tgatcgttgg gaaccggagc
5461 tgaatgaagc cataccaaac gacgagcgtg acaccacgat gcctgtagca atggcaacaa
5521 cgttgcgcaa actattaact ggcgaactac ttactctagc ttcccggcaa caattaatag
5581 actggatgga ggcggataaa gttgcaggac cacttctgcg ctcggccctt ccggctggct
5641 ggtttattgc tgataaatct ggagccggtg agcgtgggtc tcgcggtatc attgcagcac
5701 tggggccaga tggtaagccc tcccgtatcg tagttatcta cacgacgggg agtcaggcaa
5761 ctatggatga acgaaataga cagatcgctg agataggtgc ctcactgatt aagcattggt
5821 aactgtcaga ccaagtttac tcatatatac tttagattga tttaaaactt catttttaat
5881 ttaaaaggat ctaggtgaag atcctttttg ataatctcat gaccaaaatc ccttaacgtg
5941 agttttcgtt ccactgagcg tcagaccccg tagaaaagat caaaggatct tcttgagatc
6001 ctttttttct gcgcgtaatc tgctgcttgc aaacaaaaaa accaccgcta ccagcggtgg
6061 tttgtttgcc ggatcaagag ctaccaactc tttttccgaa ggtaactggc ttcagcagag
6121 cgcagatacc aaatactgtc cttctagtgt agccgtagtt aggccaccac ttcaagaact
6181 ctgtagcacc gcctacatac ctcgctctgc taatcctgtt accagtggct gctgccagtg
6241 gcgataagtc gtgtcttacc gggttggact caagacgata gttaccggat aaggcgcagc
6301 ggtcgggctg aacggggggt tcgtgcacac agcccagctt ggagcgaacg acctacaccg
6361 aactgagata cctacagcgt gagctatgag aaagcgccac gcttcccgaa gggagaaagg
6421 cggacaggta tccggtaagc ggcagggtcg gaacaggaga gcgcacgagg gagcttccag
6481 ggggaaacgc ctggtatctt tatagtcctg tcgggtttcg ccacctctga cttgagcgtc
6541 gatttttgtg atgctcgtca ggggggcgga gcctatggaa aaacgccagc aacgcggcct
6601 ttttacggtt cctggccttt tgctggcctt ttgctcacat gttctttcct gcgttatccc
6661 ctgattctgt ggataaccgt attaccgcct ttgagtgagc tgataccgct cgccgcagcc
6721 gaacgaccga gcgcagcgag tcagtgagcg aggaagcgga aga
```

### EXAMPLES

### Example 1

### Viral Production

An expression cassette was designed for expression of the gene of interest as a fusion comprising from N to C terminus: (i) the human tissue plasminogen activator leader sequence (ii) a cloning site for receipt of the gene of interest (iii) a V5 epitope tag (iv) the IMX313 adjuvant sequence. This cassette was generated by gene synthesis by Geneart AG.

Antigens, or portions of antigens, were selected. Human codon optimised sequences expressing the antigens of interest were also synthesised by Geneart AG, flanked by restriction sites suitable for in-frame insertion into the expression cassette. This was accomplished between HindIII and BamHI sites using conventional techniques.

The sequences of the cassettes expressing the EsxA and BitC fusion proteins are provided in SEQ IDs number 35 and 36, respectively. The initiator is indicated in bold and underlined. The cassettes were then cloned into two separate vectors between Acc65I and NotI sites, for the purpose of adenoviral and Modified Vaccinia Ankara production.

### Example 2

### Adenoviral Production

The cassette of Example 1 was cloned into pMono2. pMono2 is a mammalian expression vector constructed in Oxford University by modification of the vector pENTR4 (Invitrogen). Modifications were made by ligation of synthetic DNA (Geneart AG) or of oligonucleotide pairs. Its design is similar to many other mammalian expression vectors, having AttL1 and AttL2 sites, a tetracycline operator repressible CMV promoter, a multicloning site, a bovine growth hormone polyadenylation signal, and E. coli origin of replication and a kanamycin resistance gene. The sequence of pMono2.BitC is provided in SEQ ID NO: 39.

Adenovirus Hu5 vectors containing the expression cassette were generated by recombination into pAd/PLDest (Invitrogen), and Adenoviruses grown in 293/Trex cells (Invitrogen).

### Example 3

### MVA Production

The cassette of Example 1 was cloned into pMVA, which is a vector designed for insertion of the antigen cassette into the TK locus of Modified Vaccinia Ankara by in vivo recombination. The sequence of the pMVA vector containing the BitC antigen cassette is provided in SEQ ID NO: 40. The antigen is driven by a short synthetic promoter, as described in Moorthy, V. S., et al. (Safety of DNA and modified vaccinia virus Ankara vaccines against liver-stage P. falciparum malaria in non-immune volunteers. Vaccine, 2003. 21(17-18): p. 1995-2002.). MVA production and purification were essentially as described in Moorthy, V. S. et al.

### Example 4

### Luciferase Immunoprecipitation

Luciferase immunoprecipitation was used to quantify serological responses against antigens. pMono2 expression vectors were constructed expressing fusions of the gene of interest and renilla luciferase. To do this, synthetic genes (see ‘Viral production’ above) were subcloned into into a cassette in pMono2, resulting in the expression of the synthetic gene as a cytosolic fusion with renilla luciferase between HindIII and BamHI. This cassette was made by a combination of gene synthesis and conventional subcloning. The DNA encoding the resulting fusions, between Acc65I and NotI, are SEQ ID NOs: 37 and 38.

Recombinant proteins were produced by transient transfection of 293 cells with pMono2 vectors expressing the above. 24 hours after transfection, cells were lysed in a buffer containing 50 mM Tris HCl, 100 mM NaCl, 1% Triton X-100, 50% glycerol, 5 mMol EDTA, and Halt Protease Inhibitor cocktail (Thermo Scientific) at the manufacturer's recommended concentrations. Activity of the lysates was determined by adding serial dilutions of the lysate (in lysis buffer) to Renilla luciferase assay buffer (Promega), and luminometric assay using a Varioskan luminometer (Fisher Scientific). Lysates were stored at −80° C. until use.

For assays, murine serum was serially diluted (dilutions of 1:50 to 1:50,000) into an assay buffer consisting of 50 mM Tris, 100 mM NaCl, 5 mM MgCl₂, 1% Triton X-100 pH 7.5. A volume of 293 cell lysate corresponding to an activity of approximately 1×10⁶ light units was mixed with the diluted serum at room temperature for 1 hour before addition to 5 μl Protein A/G UltraLink Resin (ThermoScientific) for 1 hour. Following extensive washing in assay buffer, Renilla luciferase assay buffer was added to the wells and luminescence determined.

Interferon gamma EliSpot assays: ELISPOT assays detecting interferon gamma production by peripheral blood lymphocytes were performed prior to challenge. The protocol was as described in Spencer, A. J., et al. (Fusion of the Mycobacterium tuberculosis antigen 85A to an oligomerization domain enhances its immunogenicity in both mice and non-human primates. PloS one, 2012. 7(3): p. e33555), except that stimulation was performed with a pools of peptides spanning the relevant proteins. Peptides were reconstituted in DMSO, a pool containing all peptides for the protein made, which were used at a final concentration of 5 μg/ml total peptides per well. The DMSO concentration used was less than 0.5% final.

Peptides used for EsxA and BitC are shown below:

| | Sequence (N to C | |
|---|---|---|
| Peptide | terminus) | |
| BitC.1 | ANTRHSESDKNVLTVYSP | SEQ ID NO: 41 |
| BitC.2 | DKNVLTVYSPYQSNLIRPIL | SEQ ID NO: 42 |
| BitC.3 | YQSNLIRPILNEFEKQEHVK | SEQ ID NO: 43 |
| BitC.4 | NEFEKQEHVKIEIKHGSTQV | SEQ ID NO: 44 |
| BitC.5 | IEIKHGSTQVLLSNLHNEDF | SEQ ID NO: 45 |
| BitC.6 | LLSNLHNEDFSERGDVFMGG | SEQ ID NO: 46 |
| BitC.7 | SERGDVFMGGVLSETIDHPE | SEQ ID NO: 47 |
| BitC.8 | VLSETIDHPEDFVPYQDTSV | SEQ ID NO: 48 |
| BitC.9 | DFVPYQDTSVTQQLEDYRSN | SEQ ID NO: 49 |
| BitC.10 | TQQLEDYRSNNKYVTSFLLM | SEQ ID NO: 50 |
| BitC.11 | NKYVTSFLLMPTVIVVNSDL | SEQ ID NO: 51 |
| BitC.12 | PTVIVVNSDLQGDIKIRGYQ | SEQ ID NO: 52 |
| BitC.13 | LQGDIKIRGYQDLLQPILKG | SEQ ID NO: 53 |
| BitC.14 | YQDLLQPILKGKIAYSNPNT | SEQ ID NO: 54 |
| BitC.15 | GKIAYSNPNTTTTGYQHMRA | SEQ ID NO: 55 |
| BitC.16 | TTTGYQHMRAIYSMHHRVSD | SEQ ID NO: 56 |
| BitC.17 | IYSMHHRVSDVHQFQNHAMQ | SEQ ID NO: 57 |
| BitC.18 | VHQFQNHAMQLSKTSKVIED | SEQ ID NO: 58 |
| BitC.19 | LSKTSKVIEDVAKGKYYAGL | SEQ ID NO: 59 |
| BitC.20 | VAKGKYYAGLSYEQDARTWK | SEQ ID NO: 60 |
| BitC.21 | SYEQDARTWKNKGYPVSIVY | SEQ ID NO: 61 |
| BitC.22 | NKGYPVSIVYPIEGTMLNVD | SEQ ID NO: 62 |
| BitC.23 | PIEGTMLNVDGIALVKNAHP | SEQ ID NO: 63 |
| BitC.24 | GIALVKNAHPHPKRKKLVQY | SEQ ID NO: 64 |
| BitC.25 | HPKRKKLVQYLTSRSVQQRL | SEQ ID NO: 65 |
| BitC.26 | LTSRSVQQRLVAEFDAKSIR | SEQ ID NO: 66 |
| BitC.27 | VAEFDAKSIRKDVSEQSDQS | SEQ ID NO: 67 |
| BitC.28 | KDVSEQSDQSIENLKNIPLI | SEQ ID NO: 68 |
| BitC.29 | IENLKNIPLIPKSKLPDIPH | SEQ ID NO: 69 |
| BitC.30 | PKSKLPDIPHHKFLEMIQ | SEQ ID NO: 70 |
| Esx.14 | MIKMSPEEIRAKSQSYGQGS | SEQ ID NO: 71 |
| Esx.15 | EIRAKSQSYGQGSDQIRQIL | SEQ ID NO: 72 |
| Esx.16 | SYGQGSDQIRQILSDLTRAQ | SEQ ID NO: 73 |
| Esx.17 | QIRQILSDLTRAQGEIAANW | SEQ ID NO: 74 |
| Esx.18 | DLTRAQGEIAANWEGQAFSR | SEQ ID NO: 75 |
| Esx.19 | EIAANWEGQAFSRFEEQFQQ | SEQ ID NO: 76 |
| Esx.20 | GQAFSRFEEQFQQLSPKVEK | SEQ ID NO: 77 |
| Esx.21 | EEQFQQLSPKVEKFAQLLEE | SEQ ID NO: 78 |
| Esx.22 | SPKVEKFAQLLEEIKQQLNS | SEQ ID NO: 79 |
| Esx.23 | AQLLEEIKQQLNSTADAVQE | SEQ ID NO: 80 |
| Esx.24 | KQQLNSTADAVQEQDQQLSN | SEQ ID NO: 81 |
| Esx.25 | ADAVQEQDQQLSNNFGLQ | SEQ ID NO: 82 |

### Example 5

### S. aureus Intravenous Challenge Model and Renal Challenge Model

This was performed as described in Cheng, A. G., et al. (A play in four acts: Staphylococcus aureus abscess formation. Trends in microbiology, 2011. 19(5): p. 225-32), except that (i) the challenge was performed intravenously into the tail vein, and (ii) both kidneys were homogenised and plated separately; the geometric mean of the counts for the two kidneys was taken to be the renal S. aureus load for each animal.

### Example 6

### Protection against S. aureus is provided by MVA Single Dose Regimes Expressing BitC

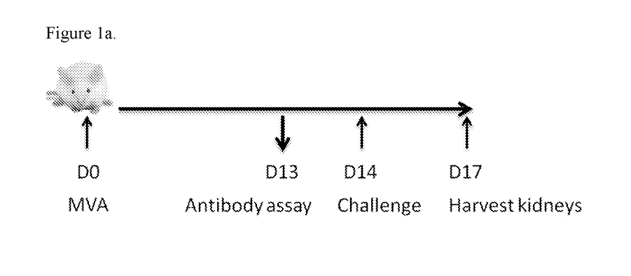

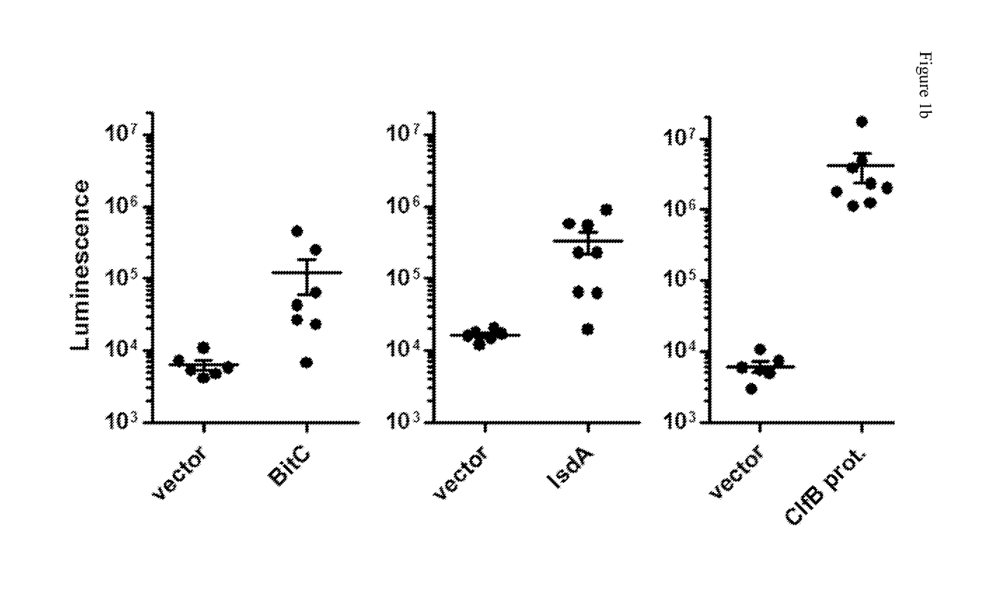

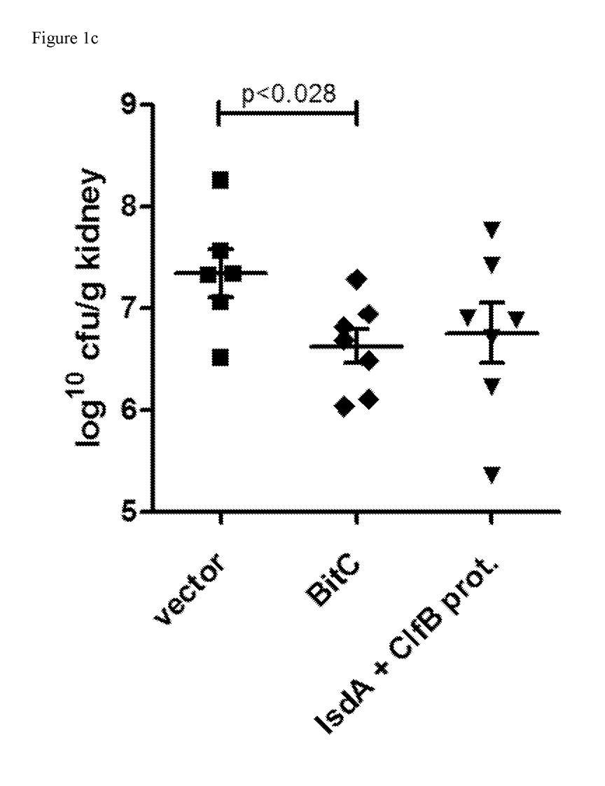

This experiment was carried out using the intravenous challenge model, which produced renal abscesses (as described above). Mice immunised with an MVA vector expressing BitC demonstrated increased antibody production and reduced renal S. aureus load following S. aureus challenge, as shown in FIG. 1 and the accompanying legend.

### Example 7

### Protection against S. aureus is provided by Adenovirus Prime, MVA Boost Regimes Expressing BitC

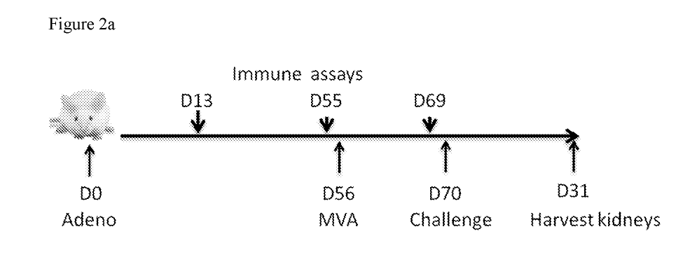

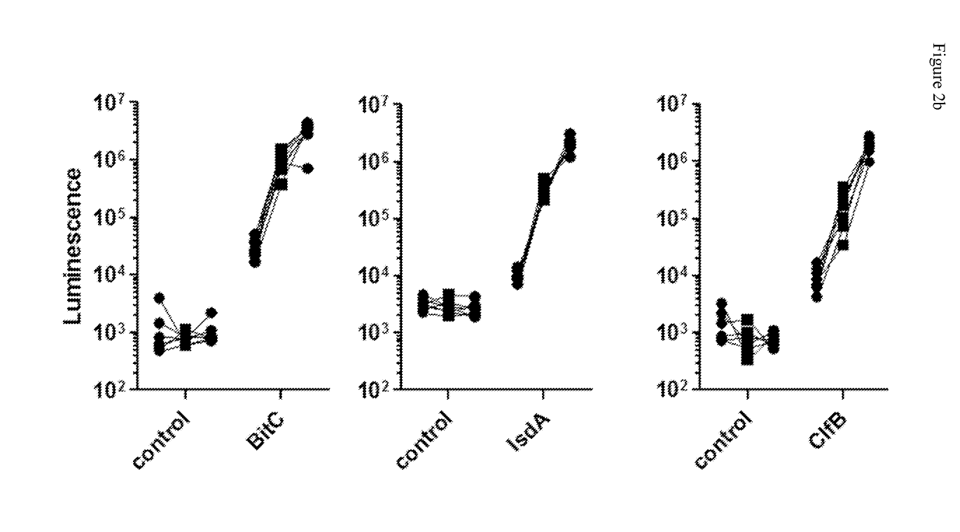

This experiment was carried out using the intravenous challenge model, which produced renal abscesses (as described above). Mice immunised with adenovirus and MVA vectors expressing BitC, using a sequential administration protocol, demonstrated increased antibody production and reduced renal S. aureus load following S. aureus challenge, as shown in FIGS. 2 and 3 and the accompanying figure legends. Both B-cell and T-cell responses were generated, and production was induced.

### Example 8

### Protection against S. aureus is provided by MVA Single Dose Regimes Expressing EsxA

This experiment was carried out using the intravenous challenge model, which produced renal abscesses (as described above). Mice immunised with an MVA vector expressing EsxA demonstrated increased antibody production and reduced renal S. aureus load following S. aureus challenge, as shown in FIG. 4 and the accompanying legend.

### Example 9

A composition of the invention is used to vaccinate infants or children as part of their routine childhood immunisation schedule. Their risk of S. aureus skin and soft tissue infection, or of invasive disease, is decreased.

### Example 10

Military recruits or prisoners, groups who are at high risk of S. aureus disease, are vaccinated on entry into the military, or to prison, using a composition of the invention. Their risk of S. aureus skin and soft tissue infection, or of invasive disease, is reduced or eliminated.

### Example 11

Individuals who are awaiting planned surgery are vaccinated in the month prior to surgery using a composition of the invention. Their risk of S. aureus wound infection, or of invasive disease, is reduced or eliminated.

### Example 12

Healthy individuals who are nasal carriers of S. aureus are vaccinated. The amount of Staphylococcus aureus in their noses is reduced or eliminated. Consequently, their personal risk of developing S. aureus disease decreases, as does the likelihood that they transmit the organism to someone else.

1. A non-replicating poxvirus vector, comprising a nucleic acid molecule encoding a Staphylococcus aureus antigen, wherein the nucleic acid molecule comprises a sequence having at least 70% sequence identity to a nucleic acid sequence selected from any one of SEQ ID NOs: 1, 2, 3, 4, 5, 6, 7, 8, 9, 10, 11, 12, 13, 14, 15, and 16.

2. The vector of claim 1, wherein the non-replicating poxvirus vector is selected from: a Modified Vaccinia virus Ankara (MVA) vector, a NYVAC vaccinia virus vector, a canarypox (ALVAC) vector, and a fowlpox (FPV) vector.

3. (canceled)

4. An adenovirus vector, comprising a nucleic acid molecule encoding a Staphylococcus aureus antigen, wherein the nucleic acid molecule comprises a sequence having at least 70% sequence identity to a nucleic acid sequence selected from any one of SEQ ID NOs: 1, 2, 3, 4, 5, 6, 7, 8, 9, 10, 11, 12, 13, 14, 15, and 16.

5. The vector of claim 4, wherein the adenovirus vector is a non-replicating adenovirus vector.

6. The vector of claim 5, wherein the adenovirus vector is selected from: a human adenovirus vector, a simian adenovirus vector, a group B adenovirus vector, a group C adenovirus vector, a group E adenovirus vector, an adenovirus 6 vector, a PanAd3 vector, an adenovirus C3 vector, a ChAdY25 vector, an AdC68 vector, and an Ad5 vector.

7. (canceled)

8. The vector of claim 1, wherein the nucleic acid molecule encoding a Staphylococcus aureus antigen encodes a Staphylococcus aureus BitC polypeptide.

9. The vector of claim 1, further comprising at least one additional nucleic acid molecule, wherein said at least one additional nucleic acid molecule encodes a Staphylococcus aureus antigen.

10. The vector of claim 9, wherein said at least one additional nucleic acid molecule comprises a sequence having at least 70% sequence identity to a nucleic acid sequence selected from any one of 1, 2, 3, 4, 5, 6, 7, 8, 9, 10, 11, 12, 13, 14, 15, 16, and 17.

11. (canceled)

12. The vector of claim 9, wherein the at least one additional nucleic acid molecule encodes a Staphylococcus aureus EsxA polypeptide.

13.-15. (canceled)

16. A composition comprising a vector according to claim 1, and a pharmaceutically-acceptable carrier.

17. The composition of claim 16, further comprising a second viral vector, wherein the second viral vector comprises a nucleic acid molecule encoding a Staphylococcus aureus antigen.

18. (canceled)

19. The composition of claim 17, wherein the first and second viral vectors are formulated separately.

20. The composition of claim 17, wherein the first and second viral vectors encode different antigens.

21. (canceled)

22. The composition of claim 17, wherein the second viral vector is selected from a non-replicating poxvirus vector and an adenovirus vector.

23. (canceled)

24. The composition of claim 22, wherein the non-replicating poxvirus vector is selected from: a Modified Vaccinia virus Ankara (MVA) vector, a NYVAC vaccinia virus vector, a canarypox (ALVAC) vector, and a fowlpox (FPV) vector.

25. (canceled)

26. The composition of claim 16, further comprising at least one Staphylococcus aureus polypeptide antigen.

27. The composition of claim 26, wherein the at least one polypeptide antigen comprises an amino acid sequence having at least 70% sequence identity to an amino acid sequence selected from SEQ ID NOs: 18, 19, 20, 21, 22, 23, 24, 25, 26, 27, 28, 29, 30, 31, 32, 33, and 34.

28. The composition of claim 26, wherein the polypeptide antigen comprises at least part of a polypeptide sequence encoded by a nucleic acid sequence of the vector.

29. The composition of claim 16, further comprising an adjuvant.

30.-49. (canceled)

50. A method of in inducing a T cell response, inducing an immune response, reducing Staphylococcus aureus carriage, or treating a Staphylococcus aureus infection, comprising administering to a subject a therapeutically effective amount of a vector according to claim 1.

51. The method according to claim 50, wherein, prior to administration, the vector is formulated as a composition comprising the vector and a pharmaceutically acceptable carrier.

52. The method according to claim 51, wherein the composition further comprises an adjuvant.
